(19) United States
(12) Patent Application Publication
(10) Pub. No.: US 2024/0240144 A1
(43) Pub. Date: Jul. 18, 2024

(54) GENERATING T CELL PRECURSORS VIA AGONIZING TUMOR NECROSIS FACTOR RECEPTOR 2

(71) Applicant: Universiteit Gent, Gent (BE)

(72) Inventors: Kai Ling Liang, Gent (BE); Tom Taghon, Aalst (BE)

(21) Appl. No.: 18/562,775

(22) PCT Filed: May 20, 2022
(86) PCT No.: PCT/EP2022/063712

§ 371 Date: Nov. 20, 2023

(30) Foreign Application Priority Data
May 25, 2021 (EP) 21175603.6

(51) Int. Cl. C12N 5/0783

(52) U.S. Cl. CPC C12N 5/0636; C12N 2501/25; C12N 2501/42; C12N 2502/1394; C12N 2506/11

(57) ABSTRACT

This disclosure relates to methods and systems for generating T cell precursors from hematopoietic stem and progenitor cells (HSPCs) in vitro. This disclosure discloses that—besides activation of the Notch signaling pathway—activation of tumor necrosis factor receptor 2 (TNFR2) present on the HSPCs is crucial to maximize the generation of T cell precursors. Hence, this disclosure relates to methods and systems, such as artificial thymic organoid systems, wherein agonists for TNFR2, such as transmembrane tumor necrosis factor (tmTNF), are used to maximally generate T cell precursors. The latter T cell precursors are useful for immune reconstitution due to transplantation or immunodeficiency disorders and may be used to generate off-the-shelf chimeric antigen receptor T cells or T cell receptor engineered T cells for immunotherapeutic purposes.

### CROSS REFERENCE TO RELATED APPLICATIONS

This application is a national phase entry under 35 U.S.C. § 371 of International Patent Application PCT/EP2022/063712, filed May 20, 2022, designating the United States of America and published as International Patent Publication WO 2022/248354 A1 on Dec. 1, 2022, which claims the benefit under Article 8 of the Patent Cooperation Treaty to European Patent Application Serial No. 21175603.6, filed May 25, 2021.

### TECHNICAL FIELD

This disclosure relates to methods and systems to generate T cell precursors from hematopoietic stem and progenitor cells (HSPCs) in vitro. This disclosure indeed discloses that—besides activation of the Notch signaling pathway—activation of tumor necrosis factor receptor 2 (TNFR2) present on the HSPCs is crucial to maximize the generation of T cell precursors. Hence, this disclosure relates to methods and systems, such as artificial thymic organoid systems, wherein agonists for TNFR2, such as transmembrane tumor necrosis factor (tmTNF), are used to maximally generate T cell precursors. The latter T cell precursors are useful for immune reconstitution due to transplantation or immunodeficiency disorders and may be used to generate off-the-shelf chimeric antigen receptor T cells or T cell receptor engineered T cells for immunotherapeutic purposes.

### BACKGROUND

Generation of human T cell precursors from hematopoietic stem and progenitor cells is dependent on activation of Notch signaling. In vitro, this is achieved through a culture system in which Notch ligands are provided as immobilized ligands or through expression by stromal cells [1-3]. Others have tried to enhance the in vitro generation and expansion of human T cell precursors by introducing additional cytokines to the culture system such as tumor necrosis factor (TNF) [4-7].

Two forms of TNF exist: the membrane-bound form (transmembrane TNF: tmTNF) and the soluble form (sTNF) [8,9]. Both tmTNF and sTNF can bind to and activate TNF receptor 1 (TNFR1, also known as CD120a). However, only tmTNF is capable to activate TNF receptor 2 (TNFR2, also known as CD120b) [10,11]. Activation of TNFR1 and TNFR2 mediate common as well as unique downstream signaling mechanisms.

Supplementation of the Notch ligand based culture system with sTNF was shown to enhance the generation of human T cell precursors. However, different groups have reported discrepancies with respect to the optimal dose and the duration of sTNF application [5-7]. Furthermore, sTNF-mediated activation of TNFR1 has been demonstrated to regulate the fate of human HSPCs through promoting their differentiation, rather than impacting self-renewal [12,13]. So, the enhanced generation of human T cell precursors by sTNF can be attributed to the accelerated differentiation of HSPCs at the expense of their maintenance.

It is however still unclear how to further maximize the generation of T cell precursors from HSPCs.

### BRIEF SUMMARY

This disclosure relates to:
1. An in vitro method to generate T cell precursors from hematopoietic stem and progenitor cells comprising contacting the hematopoietic stem and progenitor cells with an agonist for tumor necrosis factor receptor 2.
2. An in vitro method as stated above wherein the T cell precursors are CD5⁺ and CD7⁺ T cell precursors.
3. An in vitro method as stated above wherein the hematopoietic stem and progenitor cells are derived from embryonic or induced pluripotent stem cells, cord blood, bone marrow or (mobilized) peripheral blood.
4. An in vitro method as stated above wherein the T cell precursors are chimeric antigen receptor or T cell receptor engineered T cells from primary or pluripotent stem cell-derived hematopoietic stem and progenitor cells.
5. An in vitro method as stated above wherein the agonist for tumor necrosis factor receptor 2 is transmembrane tumor necrosis factor-alpha.
6. An in vitro method as stated above wherein a pool of undifferentiated CD34+ hematopoietic stem and progenitor cells is maintained.
7. An in vitro system to generate T cell precursors comprising hematopoietic stem and progenitor cells and an agonist for tumor necrosis factor receptor 2.
8. An in vitro system as stated above which is an artificial thymic organoid system comprising stromal cells that express an agonist for tumor necrosis factor receptor 2.
9. An artificial thymic organoid system as stated above wherein the amount of the stromal cells that express an agonist for tumor necrosis factor receptor 2 is about 1% of the total amount of stromal cells.
10. An artificial thymic organoid system as stated above wherein the stromal cell that expresses an agonist for tumor necrosis factor receptor 2 is a MS5 murine bone marrow stromal cell line that expresses human transmembrane tumor necrosis factor-alpha.
11. An artificial thymic organoid system as stated above wherein the MS5 murine bone marrow stromal cell line that expresses human transmembrane tumor necrosis factor-alpha—also expresses the Notch ligand human DLL4.
12. An artificial thymic organoid system as stated above comprising both the MS5 murine bone marrow stromal cell line that expresses both human transmembrane tumor necrosis factor-alpha- and the Notch ligand human DLL4 and a MS5 murine bone marrow stromal cell line that expresses human DLL4 and wherein the amounts of both cell lines are present in the artificial thymic organoid system at a ratio of 1:99, respectively.
13. An in vitro method to generate T cell precursors as stated above wherein the latter agonist improves the development potential of the T cell precursors.

### BRIEF DESCRIPTION OF THE DRAWINGS

FIG. 1: In a dose-dependent manner, sTNF accelerates differentiation of human HSPCs into T cell precursors at the expense of their maintenance. a, Scheme of assembly for the ATOs (Artificial Thymic Organoid systems) that were treated with sTNF for 10 days or without sTNF (control). b, Absolute counts of CD45⁺ cells harvested from an ATO that was aggregated with a normalized amount of 7,500 cord blood-derived HSPCs (n=10 donors for all conditions except n=3 for treatment with 0.25 ng/ml of sTNF). c-d, Flow cytometric analysis of ATOs illustrates the CD7⁺CD5⁺ T cell precursors (c) that were generated from HSPCs, of which some remained undifferentiated and expressed CD34 (d). e, Quantification of the impact of sTNF treatment on the cell counts of T cell precursors and undifferentiated CD34⁺ HSPCs compared to the control (n=10 donors for all conditions except n=3 for treatment with 0.25 ng/ml of sTNF). f-g, Flow cytometric analysis illustrates the HLA-DR expression profiles of CD1a-expressing T cell precursors (f) and quantification of the cellular fractions that were positive (g) (n=10 donors for all conditions except n=3 for treatment with 0.25 ng/ml of sTNF). Mean±s.d. of three independent experiments; Mixed-effects analysis with Dunnett's post-hoc test (b, g); Mixed-effects analysis with linear trend test (e).

FIG. 2: TNFR2 expression is inversely correlated with the ontogeny of human HSPCs and is transiently co-expressed with CD7 during early T cell development. a-b, ATOs were assembled using HSPCs derived from human fetal liver, cord blood or adult buffy coats, and were harvested for flow cytometry analyses at day 2, 4, 7 and 10 post culture. For each ontogenetic stage, expression of TNFR1 across different time points is compared to the Fluorescence Minus One control and is shown in representative offsetting histograms (a). For each ontogenetic stage, expression of TNFR2 and CD7 across different time points is shown in representative contour plots (b). c, Quantification of the proportional changes in 4 cellular subsets that have differential expression of TNFR2 and CD7, as shown in (b), across different time points. ATOs from each ontogenetic stage were assembled and analyzed independently. Fetal liver (n=2 donors; mean), cord blood (n=2 donors; mean) and buffy coat (n=4 donors; mean±s.d.); One-way ANOVA test with linear trend test for each cellular subsets (c). d, Expression of CD5 on the 4 cellular subsets of buffy coat-ATOs at day 10 post culture is shown in representative offsetting histograms.

FIG. 3: Deprivation of IL-7 hampers the development of TNFR2⁺CD7⁺ cells in ATO culture. a-b, At day 10 post culture, cord blood-ATO (lin⁻CD34⁺CD38⁻; n=3 donors) supplemented with and without IL-7 were immunophenotyped for the expression of TNFR2 and CD7 (a), and changes in the 4 cellular subsets were quantified (b). The subset comprised of TNFR2⁺CD7⁺ cells were examined further for the expression of CD5 and CD123. Mean±s.d. of one experiment; Two-way ANOVA test with Šidák's multiple comparisons test (b); IL-7, interleukin 7.

FIG. 4: TNFR2 expression on ex vivo human thymic progenitors increases during T-lineage specification but decreases in the subsequent commitment stage. a, Gating strategy to identify unspecified (1), T-lineage specified (2) and T-lineage committed (3) ex vivo human thymic progenitors. b, Expression of TNFR2 on the 3 cellular subsets identified in (a) is shown in representative offsetting histograms. c, Quantification of the median fluorescent intensity of TNFR2 as shown in (b) (n=4 donors). Mean±s.d. of one experiment; One-way ANOVA test with Tukey's post-hoc test (c); lin, lineage; MFI, median fluorescent intensity.

FIG. 5: Generation of MS5 cell line that co-expresses human transmembrane TNF (tmTNF) and DLL4 (MS5-tmTNF/DLL4). 6 weeks after sorting based on co-expression of GFP (reporter for DLL4-encoding construct) and BFP (reporter for TNF-encoding construct), expression of human tmTNF and DLL4 on transduced MS5 cells were examined by flow cytometry. MS5 parental cell line served as a negative control. MS5-DLL4 cell line served as a positive control for DLL4 expression.

FIG. 6: Spatially restricted tmTNF signal enhances generation of human T cell precursors and maintains a pool of undifferentiated CD34⁺ HSPCs. a, Scheme of ATO assembly using MS5-DLL4 (control) or MS5-tmTNF/DLL4, or a combination of both at different ratios. The ATOs were analyzed by flow cytometry after 10 days of culture. b, Absolute counts of CD45⁺ cells harvested from an ATO that was aggregated with a normalized amount of 7,500 cord blood-derived HSPCs (n=4 donors). c-d, Flow cytometric identification of CD7⁺CD5⁺ T cell precursors (c) that were generated from HSPCs, of which some remained undifferentiated and expressed CD34 (d). e, Quantification of the impact of tmTNF signal intensity on the cell counts of T cell precursors and undifferentiated CD34⁺ HSPCs compared to the control (n=4 donors). f-g, Flow cytometric analysis of the HLA-DR expression profiles of CD1a-expressing T cell precursors (f) and quantification of the cellular fractions that were positive (g) (n=4 donors). Mean±s.d. of two independent experiments; One-way ANOVA test with linear trend test (e); Friedman test with Dunnett's post-hoc test (g).

FIG. 7: Single-cell RNA sequencing (scRNA-seq) of CD45⁺ differentiating cells derived from the control and the TNF-activated ATOs (sTNF at 0.25 ng/ml or tmTNF at 1%) at day 10 post culture. a-b, Following quality control analyses and dimensionality reduction analysis, 25 distinct cell clusters were identified and annotated for their cell types based on their aggregated cluster-level gene expression profiles. Dot plot showing the expression of cell type-specific marker genes by each cluster (a) as indicated by gray scaling and size (b). c, A summary of the different populations of hematopoietic cells identified in these clusters. d, The frequency of all the identified hematopoietic populations (top) and the relative distribution of cells derived from different ATO conditions in each of these populations (bottom). e, Expression of specific TNFR1- or TNFR2-mediated NF-κB pathway genes was probed in all the identified hematopoietic populations and their respective signaling strength was indicated by the value of a ratio. f, Breakdown of the ratio of signaling mediated by TNFR1 over TNFR2 in the hematopoietic populations derived from different ATO conditions. g, Differential gene expression analysis (DGE) was performed on hematopoietic populations derived from the control and the TNF-activated ATOs (sTNF or tmTNF). The number of statistically significant differentially expressed genes found in the TNF-activated ATOs is illustrated by Venn diagrams. Cord blood HSPCs derived from 4 donors were used to assemble all ATOs of different conditions.

FIG. 8: Human T cell precursors derived from sTNF-treated ATOs are impaired in the generation of CD4⁺CD8b⁺ double positive (DP) T cells. a, Overview of the experimental design where human T cell precursors derived from different conditions (n=2 donors) were examined for their maturation at later developmental stages. b, Flow cytometry analyses at day 13 post secondary culture to identify DP T cells. c, Quantification of DP T cells generated from control-, sTNF- and tmTNF-derived human T cell precursors. d-e, Flow cytometry analyses at day 25 post secondary culture to identify CD3⁺TCRαβ⁺ and CD3⁺TCRγδ⁺ T cells (d) and their quantification (e). Changes in the development of relatively mature T cells from individual donor across different experimental conditions were indicated by the connecting line (c, e).

FIG. 9: EHD2-scTNFR2 is a TNFR2-selective agonist and promotes development of human T cell precursors. a, At day 10 post culture, buffy coat-ATO (lin⁻CD34⁺; n=4 donors), without (control) or with 10 ng/ml of EHD2-scTNFR2 treatment, were analyzed by flow cytometry to identify CD7⁺CD5⁺ T cell precursors and CD7⁻CD5⁻CD34⁺ undifferentiated HSPCs. b-c, Quantification of the impact of EHD2-scTNFR2 treatment on the generation of T cell precursors (b) and the maintenance of undifferentiated HSPCs (c) compared to the control. Mean±s.d. of one experiment; One sample two tailed t test (b).

### DETAILED DESCRIPTION

This disclosure thus relates to the selective targeting of TNFR2 in order to maximize the generation of T cell precursors from HSPCs. Indeed, this disclosure surprisingly shows that agonists for tumor necrosis factor receptor 2, in contrast to agonists for tumor necrosis factor receptor 1, allows to maintain a pool of immature HSPCs that allows continuous generation of T cell precursors. The latter continuous generation results in a significantly increased yield of T cell precursors per HSPC compared to controls and thus further maximizes the generation of T cell precursors from HSPCs. Previous findings have demonstrated that sTNF increases the generation of T cell precursors. This disclosure discloses that this impact is mediated by the activation of TNFR1 and results in a decrease in the pool of immature HSPCs. In addition, this disclosure revealed clear differences of the impact of TNFR1 versus TNFR2 activation on the intrinsic properties of the resulting T cell precursors.

Hence, this disclosure relates in first instance to an in vitro method to generate T cell precursors from HSPCs comprising contacting the HSPCs with an agonist for TNFR2.

The terms ‘to generate T cell precursors from HSPCs’ relate to differentiation of HSPCs into precursors that have been specified to T-lineage development. Phenotypically, HSPCs are defined by their expression of CD34 and lack of expression for markers that represent lineage-specific differentiated cells of particular hematopoietic lineages [14]. Hence, HSPCs are immature and, depending on the environmental cues, can give rise to blood cells of different lineages. HSPCs are heterogeneous and consist of the most primitive hematopoietic stem cells that are capable of self-renewal and their immediate progeny that lack stem cell self-renewal function but remain multipotent. Indeed, most of the stem cells divide asymmetrically (i.e., one stem cell gives rise to one stem cell and one immediate progeny). As such, the stem cell pool is maintained while the immediate progeny continues to differentiate. Ontogenetically, HSPCs emerge during embryogenesis and give rise to the adult hematopoietic system. Recapitulation of some of the processes during embryonic hematopoietic development allows us to generate HSPC-like cells from human embryonic or induced pluripotent stem cells in vitro [15]. Although these HSPC-like cells are multipotent, they are unable to engraft and reconstitute hematopoiesis in vivo. Alternatively, primary human HSPCs can be isolated from cord blood, bone marrow or mobilized peripheral blood ex vivo. T cell development takes place in the thymus where it is constantly seeded by HSPCs originating from the bone marrow. Under the influence of Notch signaling, these thymus seeding progenitors undergo stepwise differentiation to become T cell precursors that eventually give rise to multiple distinct cell subtypes such as CD4⁺ and CD8⁺ α/β T cells, γ/δ T cells and regulatory T cells [16]. During this multi-step differentiation process, cells migrate throughout the thymus where they receive the appropriate site- and stage-specific signals through cellular contact with stromal cells in distinct thymic microenvironments. The mature naïve T cells then emigrate from the thymus to peripheral lymphoid organs to function as central mediators of the immune system.

More specifically, the term ‘T cell precursor’ relates to ‘CD7⁺CD5⁺ T cell precursor.’ CD7 is a target of Notch signaling and expression of CD7 marks the specification of developing HSPCs to T lineage development [17]. However, expression of CD7 alone is insufficient to define T cell precursors as ex vivo thymic and in vitro-generated dendritic cell precursors also express CD7 [18]. Hence, T cell precursors are defined by their co-expression of CD5, which is a lymphoid marker. Subsequent expression of CD1a by CD7⁺CD5⁺ T cell precursors indicate their full commitment to T lineage development in vivo [1].

With the terms ‘contacting HSPCs with an agonist for TNFR2’ are meant bringing HSPCs that express TNFR2 into close contact with an agonist for TNFR2 so that the agonist can bind to—and activate TNFR2.

With the term ‘agonist for TNFR2’ is meant any compound or chemical that binds to TNFR2 and activates the receptor to produce a biological response. The latter biological response being the conversion of HSPCs into T cell precursors. Non-limiting examples of agonists for TNFR2 are tmTNF, TNF muteins such as EHD2-scTNFR2 (19) and agonistic antibodies [20].

This disclosure further relates to an in vitro method as described above wherein the T cell precursors can be generated from primary or pluripotent stem cell-derived HSPCs that have been genetically engineered in order to give rise to mature T cells that bear a chimeric antigen receptor (CAR) or antigen-specific T cell receptor (TCR). In recent years, T cell based immunotherapy emerged as a successful therapy for treating cancers. CAR T cells are able to recognize and target surface antigens independent of HLA whereas TCR-redirected T cells targets intracellular antigens in a HLA-dependent manner. In both approaches, engineered T cells can be allogeneic (derived from healthy donors) or autologous (derived from patients). The former requires further genetic modification in order to inactivate their alloreactivity. The use of autologous engineered T cells mitigate this problem but is limited by the time and expense to manufacture patient-specific T cell products [21]. To generate off-the-shelf universal CAR/TCR T cells, current research aim to derive engineered T cells from CAR/TCR-transduced HSPCs or pluripotent stem cells because they are non-alloreactive due to developmental allelic exclusion of endogenous TCR expression [22,23].

This disclosure further relates to an in vitro method as described above wherein a pool of undifferentiated CD34⁺ hematopoietic stem and progenitor cells is maintained. This ‘pool that is maintained’ is the main difference compared to the application of sTNF since sTNF accelerates differentiation of HSPCs into T cell precursors at the expense of proliferation. As described earlier, HSPCs contain hematopoietic stem cells that are capable of self-renewal and multi-lineage differentiation, as well as their immediate progeny that has the same properties but lost self-renewal capacity and that is characterized with a more restricted proliferative capacity. Therefore, maintenance of the pool of immature HSPCs allows continuous generation of T cell precursors. This is reflected in the application of an agonist for tumor necrosis factor receptor 2 where the number of T cell precursors generated was significantly increased compared to the control (yield of T cell precursors per HSPC). In contrast, sTNF application exhausts the pool of HSPCs by promoting faster differentiation toward T cell precursors that results in a smaller yield of T cell precursors per HSPC.

In another embodiment, this disclosure relates to any in vitro system to generate or produce T cell precursors, such as, for example, an artificial thymic organoid (ATO) system, that comprises HSPSc and an agonist for tumor necrosis factor receptor 2. The ATO system employs a simple compaction reaggregation technique, by which stromal cells that express an agonist for TNFR2 are aggregated with HSPCs by centrifugation and subsequently deployed on a cell culture insert at the air-fluid interface [22]. The three-dimensional organoid cultures allow HSPCs to develop in a thymus-like microenvironment. In contrast to the ATO system, the OP9-DLL1 co-culture technique (as another example of an in vitro system to generate t cell precursors) is a two-dimensional system where HSPCs are cultured on a monolayer of stromal cells [24,25]. Stromal cells from both the ATO and OP9-DLL1 systems are engineered to express Notch ligands. Other in vitro systems that are stromal-free involve culture of HSPCs on a surface immobilized with synthetic Notch ligands [2,3].

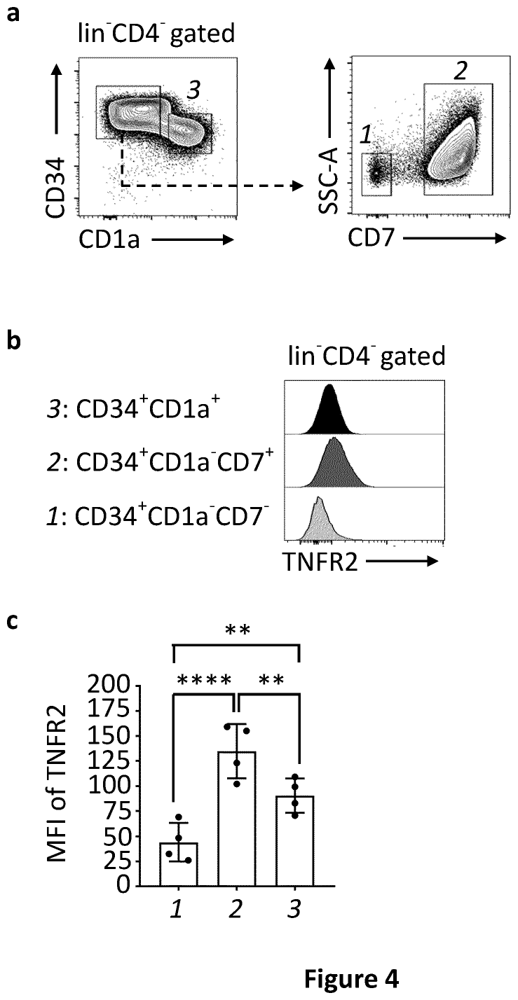

This disclosure more specifically relates to an artificial thymic organoid system wherein the amount of the stromal cells that express an agonist for tumor necrosis factor receptor 2 (TNFR2) is about 1% of the total amount of stromal cells. With the term ‘about 1%’ is meant 0.5, 0.6, 0.7, 0.8, 0.9, 1, 1.1, 1.2, 1.3, 1.4 or 1.5%, or, ranging between 0.5 and 1.5%. An ATO is typically assembled by aggregation of 7,500 HSPCs with 150,000 stromal cells [22]. This disclosure discloses that the presence of 1% TNFR2-agonist expressing stromal cells (1,500 per ATO) increases significantly the number of T cell precursors generated while maintaining the number of undifferentiated CD34⁺ HSPCs compared to the control (FIG. 4E), which in turn results in a higher yield of T cell precursors per HSPC. Furthermore, this amount of TNFR2-agonist expressing stromal cells (about 1% or 1,500 per ATO) did not lead to aberrant upregulation of HLA-DR expression on the CD1a-expressing T cell precursors (FIGS. 4F and 4G). Increase expression of HLA-DR is an indication of TNFR1 activation [26]. This indicates that the agonist targets TNFR2 selectively when it is expressed by about 1% of the total amount of stromal cells per ATO.

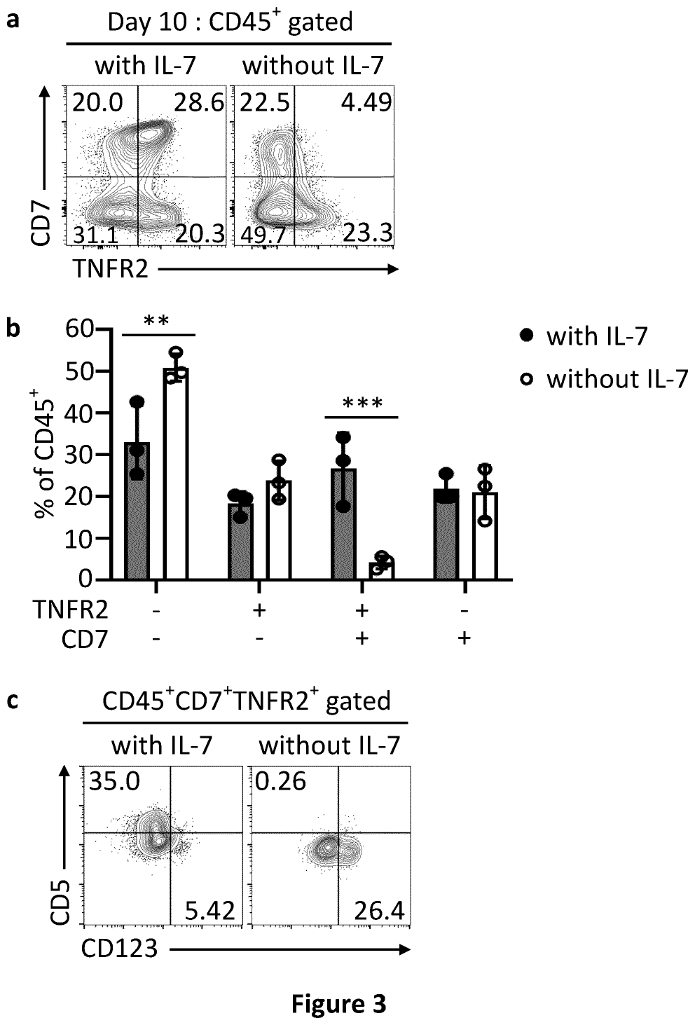

This disclosure further relates to an artificial thymic organoid system as described above wherein the stromal cell that expresses an agonist for tumor necrosis factor receptor 2 is a MS5 murine bone marrow stromal cell line that expresses human transmembrane tumor necrosis factor-alpha (MS5-tmTNF). The MS5 cell line was established by irradiation of the adherent cells in long-term murine bone marrow cultures [27]. It is commercially available from Leibniz Institute DSMZ-German Collection of Microorganisms and Cell Cultures, Braunschweig Germany (DSMZ no. ACC 441). To generate MS5-tmTNF, MS5 cells were transduced with a retroviral vector encoding the nucleotide coding sequence of human TNF (NM_000594.4: 178-879 bp) and blue fluorescent protein (FIG. 3). In the ATO system, the MS5 cell line was shown to perform better than OP9 cell line to support robust T cell development [22]. It should be clear however that other stromal cell lines than MS5 and OP9 can be used as well.

This disclosure further relates to an artificial thymic organoid system as described above wherein the MS5 murine bone marrow stromal cell line that expresses human transmembrane tumor necrosis factor-alpha—also expresses the Notch ligand human DLL4 (MS5-tmTNF/DLL4). DLL4 is a non-limiting example of one of the four Notch ligands (DLL1, DLL4, JAG1 and JAG2) that are expressed in the thymus. However, only DLL1, DLL4 and JAG2 can induce T-lineage differentiation in human HSPCS [28]. Among them, DLL4 induces the strongest signal strength through NOTCH1 binding, followed by DLL1 and JAG2 [28]. In vitro, DLL1 and DLL4 are used interchangeably to support T cell precursor generation from human HSPCs [1-3,7,22-24]. To generate MS5-tmTNF/DLL4, this disclosure discloses the co-transduction of MS5 cells with two retroviral vectors. One encodes the nucleotide coding sequence of human TNF (NM_000594.4: 178-879 bp) and blue fluorescent protein, and the other encodes the nucleotide coding sequence of human DLL4 (AF253468.1) and green fluorescent protein (FIG. 3).

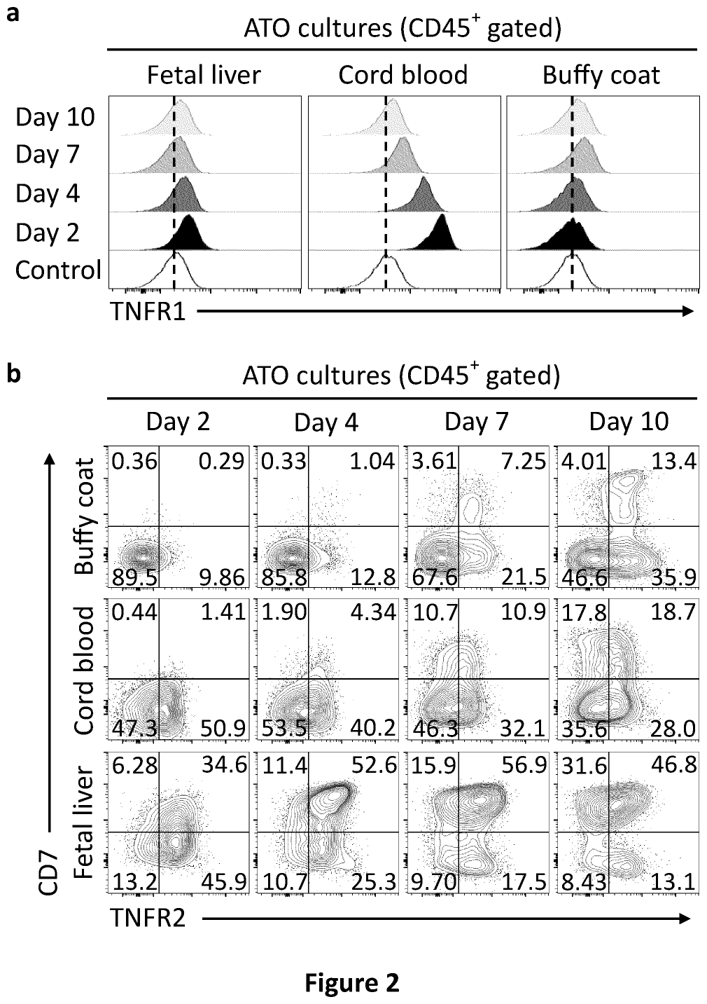

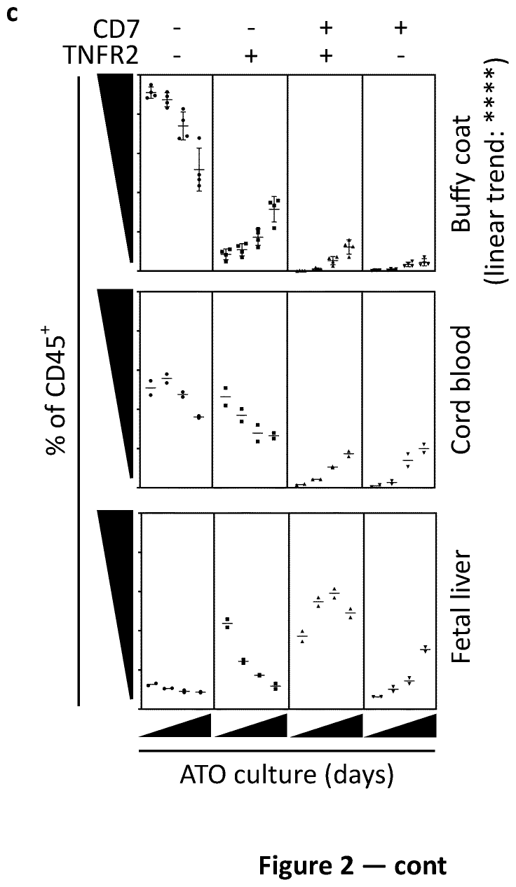

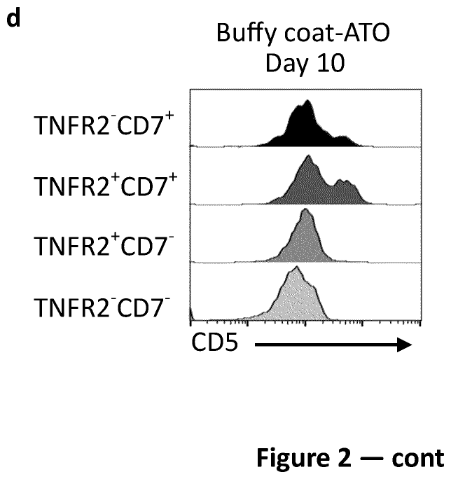

This disclosure further relates to an artificial thymic organoid system as described above comprising both the MS5 murine bone marrow stromal cell line that expresses both human transmembrane tumor necrosis factor-alpha- and the Notch ligand human DLL4 and a MS5 murine bone marrow stromal cell line that expresses human DLL4 and wherein the amounts of both cell lines are present in the artificial thymic organoid system at a ratio of about 1:99, respectively. With the term ‘a ratio of about 1:99’ is meant a ratio of 1:98.5, 1:98.6, 1:98.7, 1:98.8, 1:98.9, 1:99, 1:99.1, 1:99.2, 1:99.3, 1:99.4 or 1:99.5, or, ranging between 1:98.5 and 1:99.5. At 1:99 ratio, for example, tmTNF is presented by 1% of the total stromal cells that are dispersed throughout the three-dimensional ATO. As such, the tmTNF signal is presented to contacting HSPCs in a spatially restricted manner. This is important given that tmTNF can bind to activate both TNFR1 and TNFR2 [10]. Physiologically, HSPCs downregulate TNFR1 and upregulate TNFR2 expression as they differentiate to become T cell precursors (FIG. 2). Hence, the spatially restriction of tmTNF signal at 1% minimizes the activation of TNFR1 and allows selective targeting of TNFR2 on differentiating HSPCs in order to maximize the generation of T cell precursors.

With the term ‘intrinsic properties of the resulting T cell precursors’ as indicated above is meant cell quality attributes that can indicate the functional activity of T cell precursors. T cell precursors must not only have the correct identify, as demonstrated through the co-expression of CD7 and CD5 by flow cytometry, they must also function appropriately in order to generate mature T cells for therapeutic applications. These cell quality attributes include molecular characterization by gene expression profiling and potency assessment by in vitro functional assays. Hence, this disclosure relates to an in vitro method to generate T cell precursors from hematopoietic stem and progenitor cells comprising contacting the hematopoietic stem and progenitor cells with an agonist for tumor necrosis factor receptor 2 wherein the latter agonist improves the development potential of the T cell precursors. Activation of TNFR1 by sTNF and TNFR2 by tmTNF both increase the generation of phenotypically similar T cell precursors and this was reflected by the common genes that are differentially expressed in these cells in comparison to the control. However, TNFR1- and TNFR2-agonized T cell precursors are not intrinsically identical given that they also express unique differentially expressed genes. Furthermore, in consistence with the differences identified in their transcriptomes, TNFR2-agonized T cell precursors are more efficient than TNFR1-agonized T cell precursors to further differentiate into CD4⁺CD8⁺ double positive T cells. Hence, increased generation of T cell precursors via agonizing TNFR2 do not compromise the cell quality.

### Examples

### Materials and Methods

### Retroviral Constructs

The LZRS-DLL4-IRES-EGFP plasmid has been described by ref [29,30]. The full length coding sequence of human TNF (NM_000594.4: 178-879 bp) was cut from the customized IDT gBlock gene fragment using BamHI and EcoRI restriction enzymes, and ligated into LZRS-IRES-BFP. TNF sequence in LZRS-TNF-IRES-BFP was validated by Sanger sequencing. Cell culture supernatant containing the retroviral particles was prepared as described previously [31].

### MS5 Parental and Transduced Cell Lines

The MS5 parental cell line has been described by ref [27,32]. MS5-DLL4 cell line has been made in-house previously [33]. To generate the MS5-TNF/DLL4 cell line, MS5 parental cells were resuspended in culture medium containing DLL4- and TNF-encoding retroviral particles, and seeded onto a tissue culture dish coated with RetroNectin reagent (Takara, Cat #T100B). Spinfection was performed at 890×g for 90 minutes at 32° C. 2 days post transduction, cells were sorted based on co-expression of GFP and BFP. Stable expression of GFP and BFP on the MS5-tmTNF/DLL4 cell line was confirmed by flow cytometry after several weeks of culture. Expression of DLL4 (BioLegend, Cat #346505, RRID: AB_2292978) and tmTNF (BioLegend, Cat #502943, RRID: AB_2562870) were also validated by flow cytometry. All cell lines were cultured in MEM a medium (Thermo Fisher Scientific, Cat #22561021) containing 100 U/mL penicillin/streptomycin (Thermo Fisher Scientific, Cat #15140122) and 10% (v/v) of fetal calf serum (GE Life Sciences, Cat #SV30160.03), and incubated in a humidified atmosphere containing 5% (v/v) CO₂ at 37° C.

### Isolation of Hematopoietic Stem and Progenitor Cells (HSPCs)

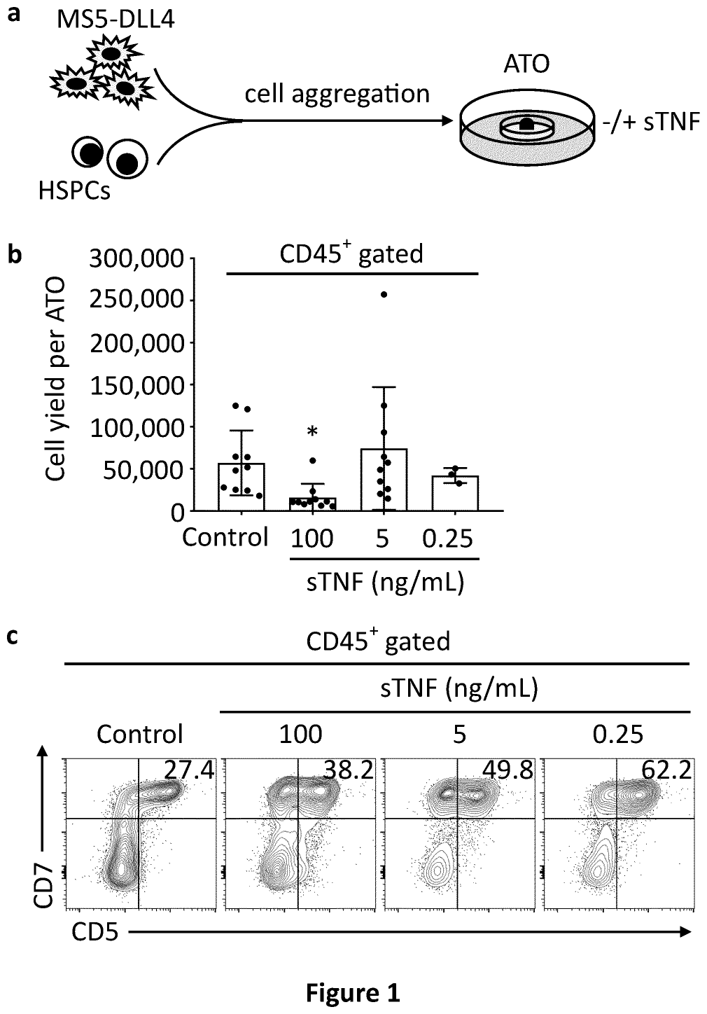

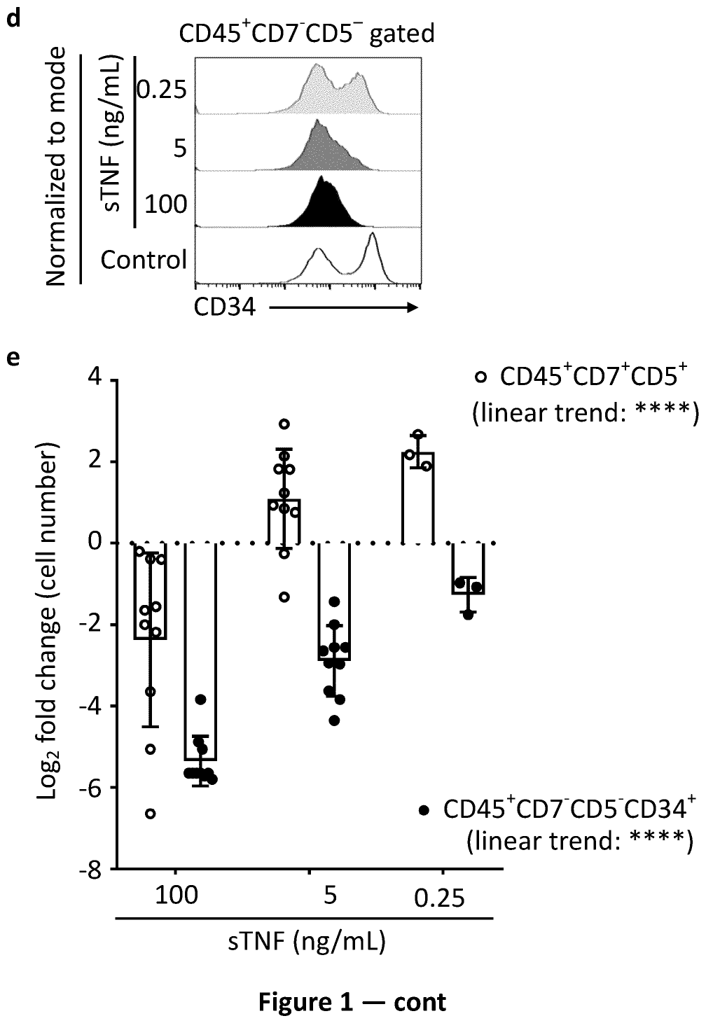

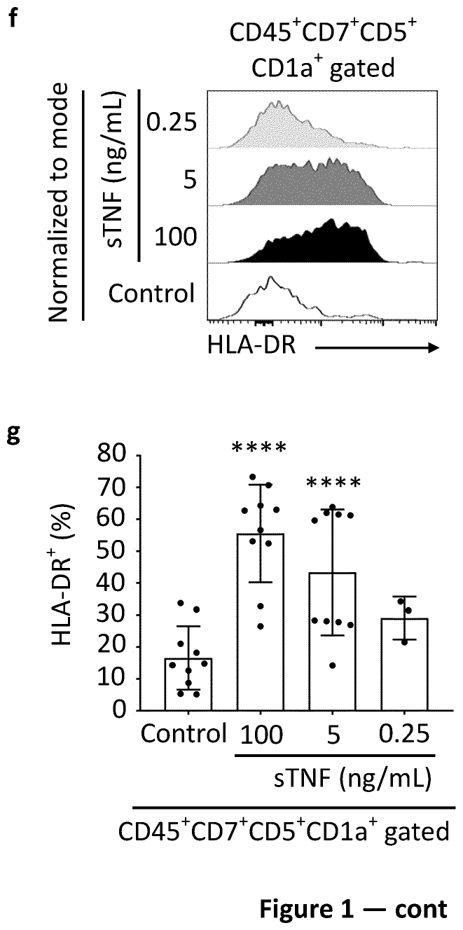

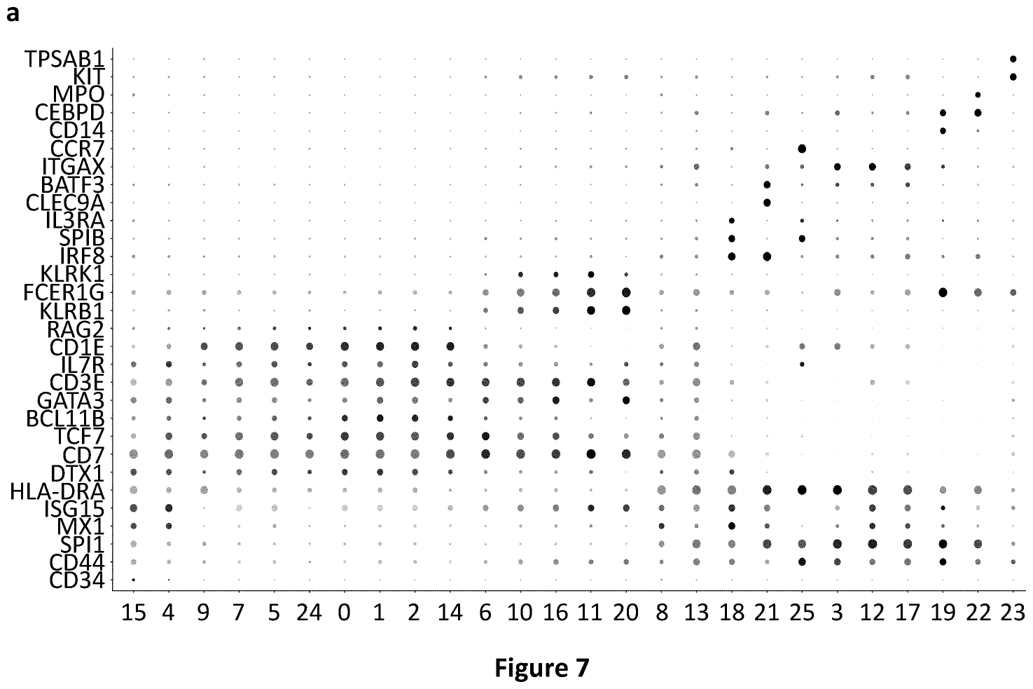

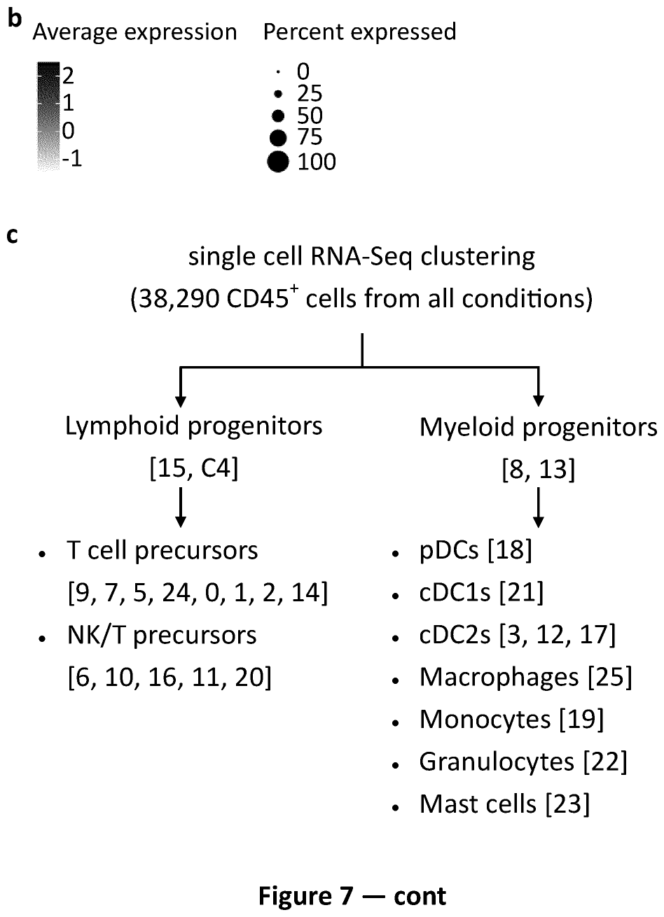

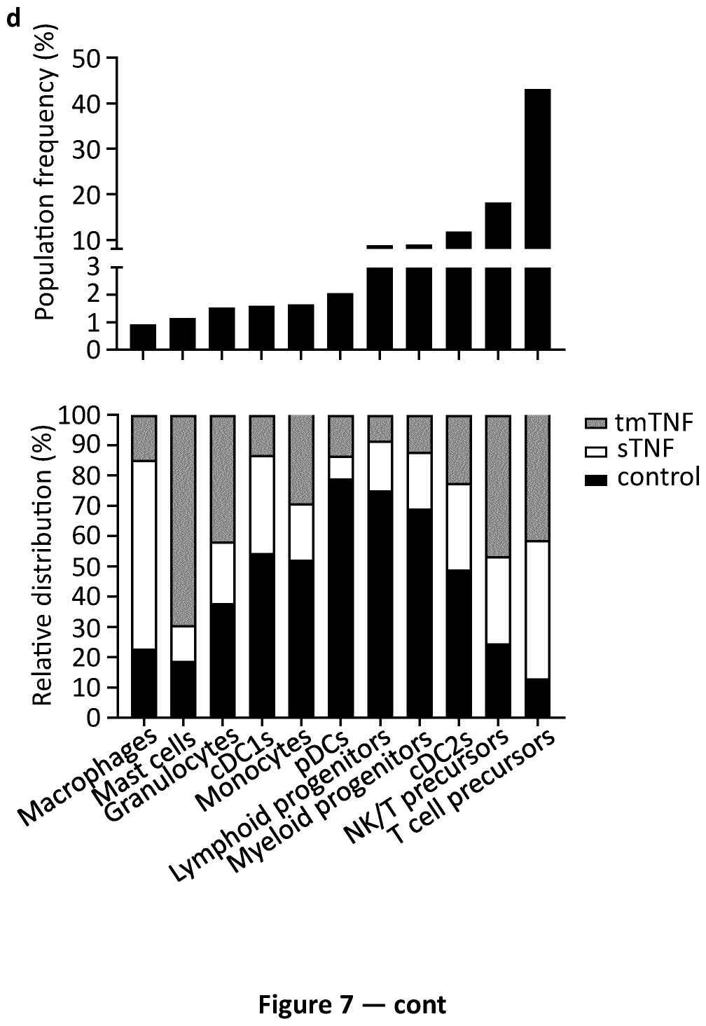

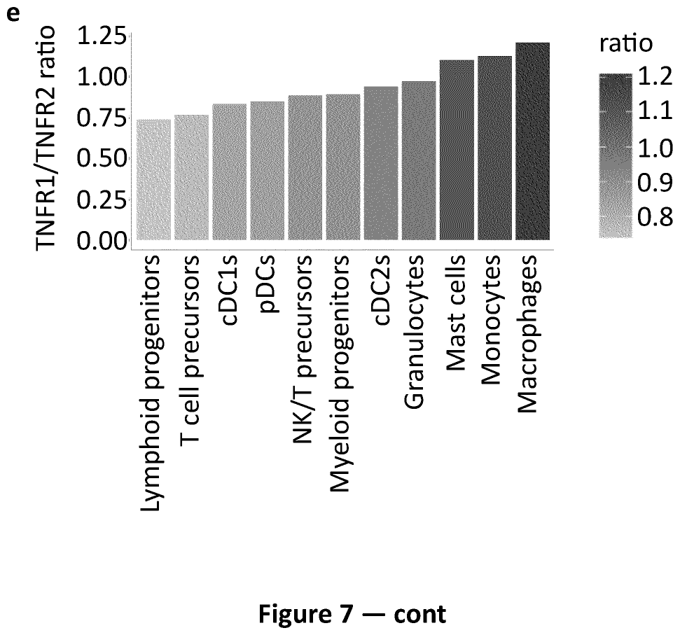

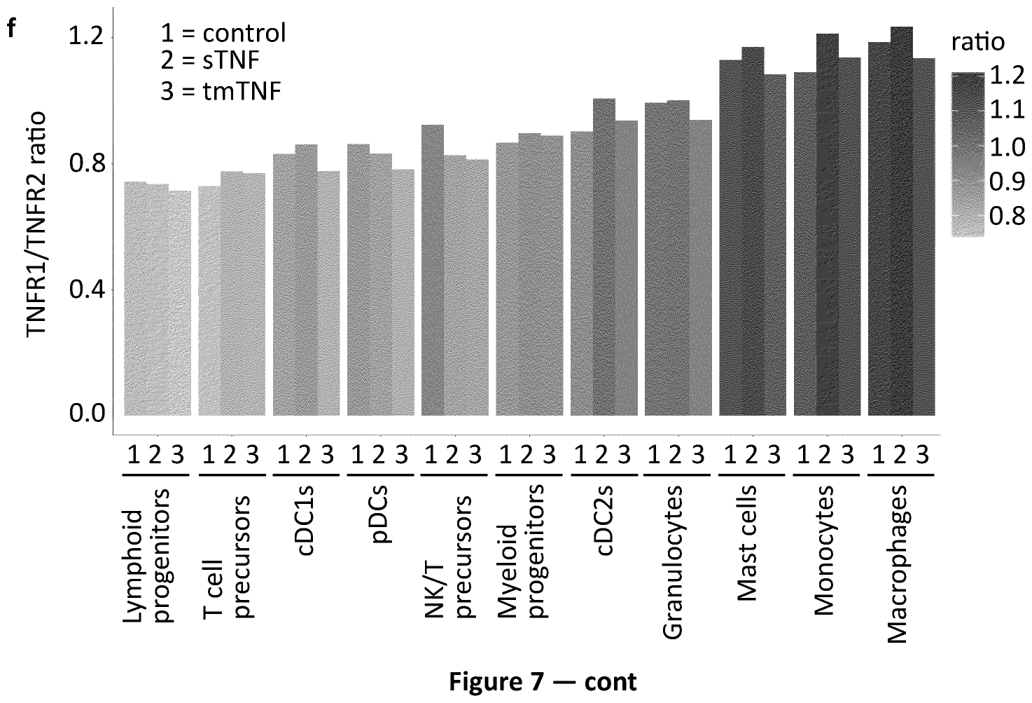

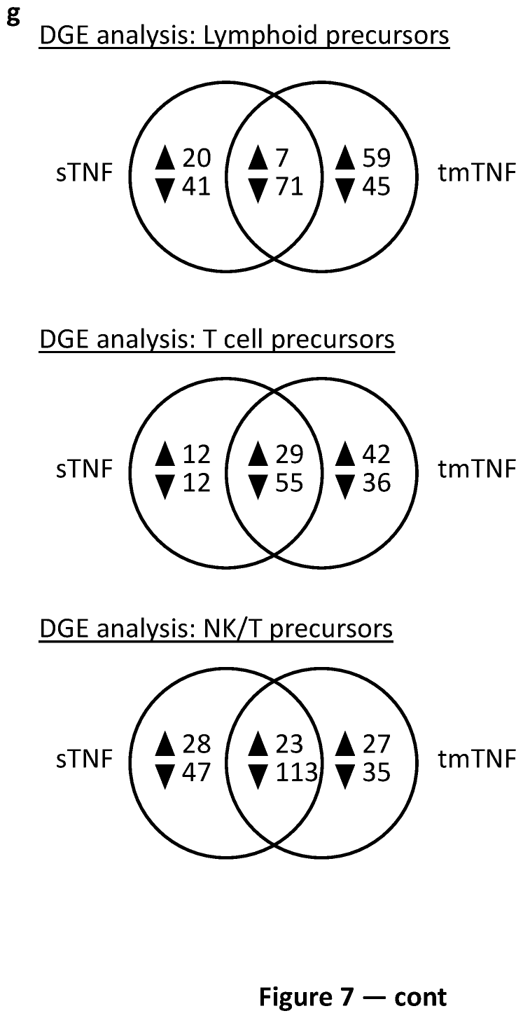

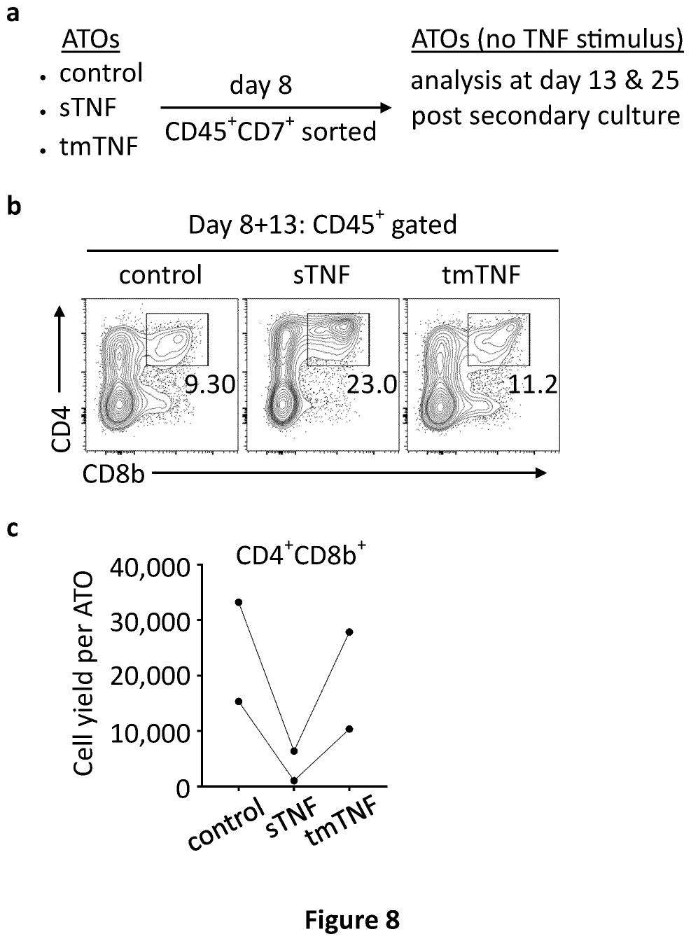

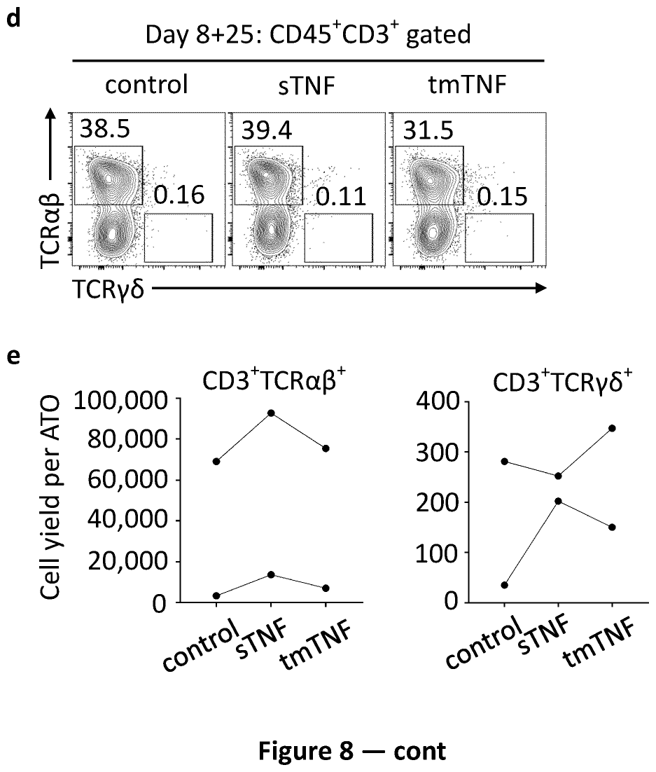

Human umbilical cord blood (CB) and adult buffy coats were used according to the guidelines of the Medical Ethical Commission of Ghent University Hospital, Belgium after informed consent had been obtained in accordance with the Declaration of Helsinki. Mononuclear cells were isolated from CB and buffy coats by Lymphoprep density gradient centrifugation (Axis-Shield, Cat #1114547). Subsequently, CD34⁺ cells from CB and buffy coats were labelled by microbeads and isolated from the mononuclear cell fraction by magnetic-activated cell sorting (Miltenyi Biotec, Cat #130-046-703). For CB, CD34-enriched cells were further stained with antibodies against CD34 (BioLegend, Cat #343510, RRID: AB_1877153), CD3 (BioLegend, Cat #300408, RRID: AB_314062), CD14 (BioLegend, Cat #301806, RRID: AB_314188), CD19 (BioLegend, Cat #302208, RRID: AB_314238), CD56 (BioLegend, Cat #362508, RRID: AB_2563924) and CD38 (BioLegend, Cat #303526, RRID: AB_10983072), and were sorted on a BD FACSAria II Cell Sorter. lin⁻(CD3⁻CD14⁻CD19⁻CD56⁻) CD34⁺CD38⁻ cord blood HSPCs were used to assemble ATOs in FIGS. 1, 2, 4 and 6 whereas lin⁻CD34⁺ cord blood HSPCs were used to assemble ATOs in FIGS. 7 and 8. For buffy coats, CD34-enriched cells were stained similarly and HSPCs that are lin⁻CD34⁺ were sorted. Mononuclear cells from human fetal liver tissues were used with approval of the Medical Ethical Commission of Ghent University Hospital, Belgium [34]. After thawing, fetal liver cells were stained with antibodies against CD34 (BioLegend, Cat #343503, RRID: AB_1731923) and CD45 (BioLegend, Cat #304028, RRID: AB_893338), and were sorted to obtain HSPCs that are CD34^{hi}CD45⁺.

### Isolation of Thymic Progenitors

Postnatal thymus was obtained from patients undergoing cardiac surgery with informed consent of parents or guardians and used with permission of and according to the guidelines of the Medical Ethical Commission of Ghent University Hospital, Belgium. Mononuclear cells were isolated from thymic total cell suspension by Lymphoprep density gradient centrifugation (Axis-Shield, Cat #1114547). Subsequently, CD34⁺ cells were labelled by microbeads and isolated from the mononuclear cell fraction by magnetic-activated cell sorting (Miltenyi Biotec, Cat #130-046-703).

### Artificial Thymic Organoids (ATOs)

ATOs were assembled and cultured as described with the exception that DLL4 instead of DLL1 was used as the Notch ligand to support T cell development. As indicated in the individual experiments, ATOs were assembled using MS5-DLL4 stromal cells or MS5-tmTNF/DLL4 stromal cells or in combination at different ratios. Per ATO, up to 7,500 HSPCs were aggregated with a total amount of 150,000 stromal cells in 5 μL of the ATO culture medium. The ATO culture medium referred to RPMI 1640 medium (Thermo Fisher Scientific, Cat #52400025) containing 4% (v/v) of B-27 Supplement (Thermo Fisher Scientific, Cat #17504044), 30 μM of ascorbic acid (Merck, Cat #A8960), 100 U/mL of penicillin/streptomycin (Thermo Fisher Scientific, Cat #15140122), 1% (v/v) of GlutaMAX Supplement (Thermo Fisher Scientific, Cat #35050061), 5 ng/mL of IL-7 (Miltenyi Biotec, Cat #130-095-364) and 5 ng/ml of FLT3-L (Miltenyi Biotec, Cat #130-096-480). 2-3 ATOs were transferred onto the membrane of a cell culture insert (Merck, Cat #PICM0RG50) that was partially submerged in 1 mL of the ATO culture medium. Medium was refreshed every 2-3 days. ATO cultures were incubated in a humidified atmosphere containing 5% (v/v) CO₂ at 37° C. Whenever indicated, ATOs were cultured and refreshed with medium without IL-7 or also containing 0.25, 5 or 100 ng/mL of soluble TNF (Miltenyi Biotec, Cat #130-094-015) or 10 ng/ml of EHD2-scTNFR2. EHD2-scTNFR2 is a TNFR2-selective TNF mutein, which consists of a covalently stabilized human TNFR2-selective (D143N/A145R) single-chain TNF (scTNFR2) fused to the dimerization domain EHD2 derived from the heavy chain domain CH2 of IgE, constituting a disulfide-bonded dimer that is, with respect to TNF domains, hexameric [19]. At indicated time point, cells were harvested for flow cytometry analyses by disrupting ATOs via forceful pipetting.

### Secondary Culture

lin⁻CD34⁺ cord blood HSPCs were assembled into ATOs in the absence (control) or the presence of TNF stimulus (sTNF at 0.25 ng/ml or tmTNF at 1%). After 8 days of culture, CD45⁺CD7⁺ cells were sorted from each condition directly into 96-well polystyrene conical bottom microplate (Thermo Fisher Scientific, Cat #249662). Single-cell sorting mode was used to accurately sort 3,500 cells into a well containing 100 μL ATO culture medium. 150,000 MS5-DLL4 stromal cells were added into each well post sorting. The microplate was centrifuged at 300×g for 5 minutes at 4° C. Following removal of supernatant, ATOs (secondary culture) were assembled and cultured as described earlier.

### Flow Cytometry

To immunophenotype differentiating HSPCs in the ATOs, cells were stained with antibodies against CD45 (BioLegend, Cat #304028, RRID: AB_893338), CD7 (BD Biosciences, Cat #561603, RRID: AB_10898348), CD5 (BD Biosciences, Cat #563381, RRID: AB_2744435), CD34 (BioLegend, Cat #343504, RRID: AB_1731852), HLA-DR (Thermo Fisher Scientific, Cat #47-9956-42, RRID: AB_1963603), TNFR1 (BioLegend, Cat #369905, RRID: AB_2650763), TNFR2 (BioLegend, Cat #358412, RRID: AB_2564396) and CD123 (Thermo Fisher Scientific, Cat #48-1239-42, RRID: AB_1548710). To immunophenotype ex vivo thymic progenitors, CD34-enriched cells were stained with antibodies against CD3 (BioLegend, Cat #300408, RRID: AB_314062), CD14 (BioLegend, Cat #301806, RRID: AB_314188), CD19 (BioLegend, Cat #302208, RRID: AB_314238), CD56 (BioLegend, Cat #362508, RRID: AB_2563924), CD4 (Biolegend, Cat #300539, RRID: AB_2562053), CD34 (Biolegend, Cat #343504, RRID: AB_1731852), CD1a (Biolegend, Cat #300110, RRID: AB_314024), CD7 (BD Biosciences, Cat #561603, RRID: AB_10898348) and TNFR2 (Biolegend, Cat #358412, RRID: AB_2564396). To identify T cell development at later stages, cells derived from ATOs were stained with antibodies against CD45 (BioLegend, Cat #304028, RRID: AB_893338), CD4 (BioLegend, Cat #300512, RRID: AB_314080), CD8b (Beckman Coulter, Cat #IM2217U), CD3 (BioLegend, Cat #300429, RRID: AB_893301), TCRαβ (BioLegend, Cat #306718, RRID: AB_10612569) and TCRγδ (BioLegend, Cat #331217, RRID: AB_2562316). Human and mouse FcR blocking reagents (Miltenyi Biotec, Cat #130-059-901 and 130-092-575) were used to minimize non-specific binding of antibodies. Precision Count Beads (BioLegend, Cat #424902) were used to obtain an absolute count of the cells. Dead cells were labeled by propidium iodide (Thermo Fisher Scientific, Cat #P3566) staining to exclude them from the flow cytometric analysis. UltraComp eBeads (Thermo Fisher Scientific, Cat #01-2222-41) were used to prepare single-color compensation controls for all antibodies whereas living and dead Jurkat cells were used as a control to compensate for the spillover of propidium iodide. Fully stained cells were measured on a BD LSR II SORP flow cytometer or a BD FACSymphony A3 Cell Analyzer. Both equipment are equipped with violet (405 nm), blue (488 nm), yellow-green (561 nm) and red (640 nm) lasers.

Single-Cell RNA Sequencing (scRNA-Seq)

Cord blood lin⁻CD34⁺ HSPCs were cultured in ATOs in the absence (control) or presence of TNF stimulus (sTNF at 0.25 ng/ml or tmTNF at 1%). At day 10 post culture, CD45⁺ cells were sorted from all conditions for scRNA-seq where libraries were prepared and sequenced according to the Chromium Single Cell Gene Expression workflow. Using CellRanger 6.0.1, the sequencing data was mapped against the GRCh38 genome. The filtered feature-barcode matrices were loaded into R. Low quality cells were identified as having less than 200 genes, more than 6000 genes (doublets) or more than 5% mitochondrial reads. Low quality genes were identified as being expressed in less than 3 cells. Both low quality cells and genes were removed. Equal number of cells were sub-sampled from all 3 ATO conditions and integrated (total: 38,439 cells) prior clustering using Seurat [35]. The UMAP method was used to visualize the cell clusters. The two smallest clusters (26: 0.22% and 27: 0.17%) were removed from the original identified 28 clusters. The remaining 26 clusters (38,290 cells: 0 to 25) were manually annotated based on cell type-specific markers genes that are differentially expressed as determined by using FindAllMarkers from Seurat. Dot plot was used to visualize of the expression of cell type-specific marker genes. To determine the strength of TNFR1- and TNFR2-mediated signaling in different hematopoietic populations, UCell was used to score two gene sets from the Molecular Signatures Database of GSEA (M27438: TNFR1-induced NFκB pathway and M27552: TNFR2-induced non-canonical NF-κB pathway) [38]. To compare the hematopoietic populations derived from the control and the TNF-activated ATOs (sTNF or tmTNF), differential gene expression analysis was performed using FindMarkers from Seurat.

### Statistical Analysis and Software

Flow cytometry data were analyzed and visualized using BD FlowJo v10. GraphPad Prism 9 was used for statistical analyses and graphing. The Gaussian distribution of experimental data (residuals) was examined visually by a Quantile-Quantile normality plot and with Shapiro-Wilk statistical tests. Based on the outcome of these normality tests, appropriate statistical tests were performed and indicated in the figure legends. All the differences detected were considered to be significant and indicated when p<0.05 (*), p<0.01 (**), p<0.001 (***) and p<0.0001 (****).

### Results

Supplementation of a Notch ligand based culture system with soluble TNF (sTNF) was shown to enhance the generation of human T cell precursors, but the different groups have reported discrepancies with respect to the optimal dose and the duration of sTNF application [5-7]. Furthermore, sTNF-mediated activation of TNFR1 has been demonstrated to regulate the fate of human HSPCs through promoting their differentiation, rather than impacting self-renewal [12,13]. So, the enhanced generation of human T cell precursors by sTNF can be attributed to the accelerated differentiation of HSPCs at the expense of their maintenance. This disclosure firstly discloses the impact of sTNF on the artificial thymic organoid (ATO) system (FIG. 1A). The ATO system used in this disclosure uses the minimum number of cytokines (IL-7 and FLT3-L at 5 ng/mL) to support in vitro human T cell development [22]. This allows to determine the true effect of sTNF on the generation of human T cell precursors without the interference from other cytokines such as SCF and TPO that are known to stimulate the proliferation of HSPCs and that are used in other in vitro systems [2,3,39]. 10 days after the culture of ATOs, it was found that sTNF treatment at 0.25, 5 and 100 ng/mL did not improve the total cell yield. In fact, 100 ng/ml of sTNF significantly decreased the total cell yield compared to the untreated control (FIG. 1B). Further examination of the cellular composition of ATOs revealed that, compared to the control, increasing dosage of sTNF treatment is inversely correlated with the increase in the proportion of CD7⁺CD5⁺ T cell precursors as a higher dose of sTNF decreases the proportion of CD34⁺ HSPCs that remains undifferentiated (FIGS. 1C-1E). Only sTNF treatment at the lowest tested dose (0.25 ng/mL) significantly increases the generation of T cell precursors (log₂ fold change: 2.25±0.40) because the pool of undifferentiated CD34⁺ HSPCs was significantly depleted but to the least extent (log₂ fold change: −1.27±0.42) compared to sTNF treatment at higher doses, hence did not improving the overall cellular output (FIG. 1E). Overall, this indicates that, in the presence of Notch signaling, HSPCs are skewed by sTNF, in a dose-dependent manner, to favor T-lineage differentiation instead of proliferation. This further shows that the previously reported positive effect of sTNF treatment at high dose on the expansion of human T cell precursors was inadvertently contributed by the presence of additional cytokines used in the assayed systems [5-7]. Surprisingly, this disclosure demonstrates that HLA-DR expression is aberrantly upregulated in the CD1a-expressing T cell precursors that are derived from sTNF-treated ATOs (FIGS. 1F and 1G). Physiologically, T cell precursors express CD1a following their full commitment to T-lineage development and have minimal expression of HLA-DR [40]. Increased expression of HLA-DR during the maturation of human dendritic cells was shown to be a unique feature of TNFR1 activation [26]. Hence, this disclosure discloses that sTNF-mediated TNFR1 activation is dose-dependent and accelerates the differentiation of human HSPCs to T cell precursors at the expense of their maintenance and/or proliferation.

TNF signaling can be exerted through activation of TNFR1 and TNFR2, and can be modulated by the changes in expression for both receptors during human aging [41]. The expression of TNFR1 and TNFR2 during early human T cell development has not been reported. Using the ATO system, this disclosure further discloses the kinetic expression of both TNF receptors on differentiating HSPCs that were derived from three human ontogenetic stages. At day 2 post culture, only cord blood-derived HSPCs expressed detectable amounts of TNFR1 compared to the Fluorescence Minus One control. Further culture revealed that TNFR1 expression was gradually downregulated as cord blood-derived HSPCs differentiate along the T cell lineage (FIG. 2A). This is in agreement with the above indicated findings that the dose-dependent sTNF-mediated activation of TNFR1 by actually restricts expansion of T cell precursors. In contrast to that, this disclosure discloses that TNFR2 expression is inversely correlated with the ontogeny of HSPCs but is induced and transiently co-expressed with CD7 expression during early T cell development (FIGS. 2B and 2C). CD7 is a target of Notch signaling that marks the specification of HSPCs to T cell lineage development [17]. Detection of CD5 expression on TNFR2⁺CD7⁺ cells confirmed their T-lineage identity (FIG. 2D), which was further corroborated by the observation that the development of TNFR2⁺CD7⁺ cells was impaired in ATO cultures without IL-7 supplementation (FIGS. 3A and 3B), resulting in expression of CD123, which is a myeloid marker [42] instead of CD5 (FIG. 3C). This disclosure validates the kinetic expression of TNFR2 observed in ATO cultures by immunophenotyping ex vivo human thymic progenitors (FIG. 4A). In contrast to CD7⁻ thymic progenitors that have the lowest TNFR2 expression, T-lineage specified CD7⁺ thymic progenitors express the highest level of TNFR2 followed by T-lineage committed thymic progenitors (FIGS. 4B and 4C). Collectively, this disclosure shows that TNF signaling is primarily mediated by TNFR2 during early human T cell development and that it works synergistically with Notch signaling to promote differentiation of HSPCs into T cell precursors.

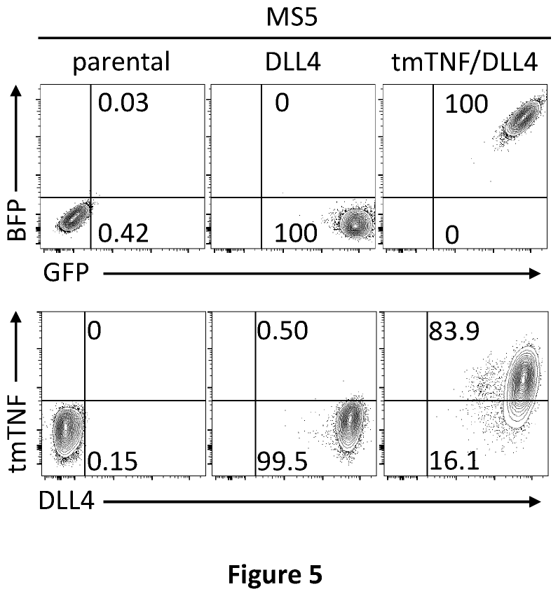

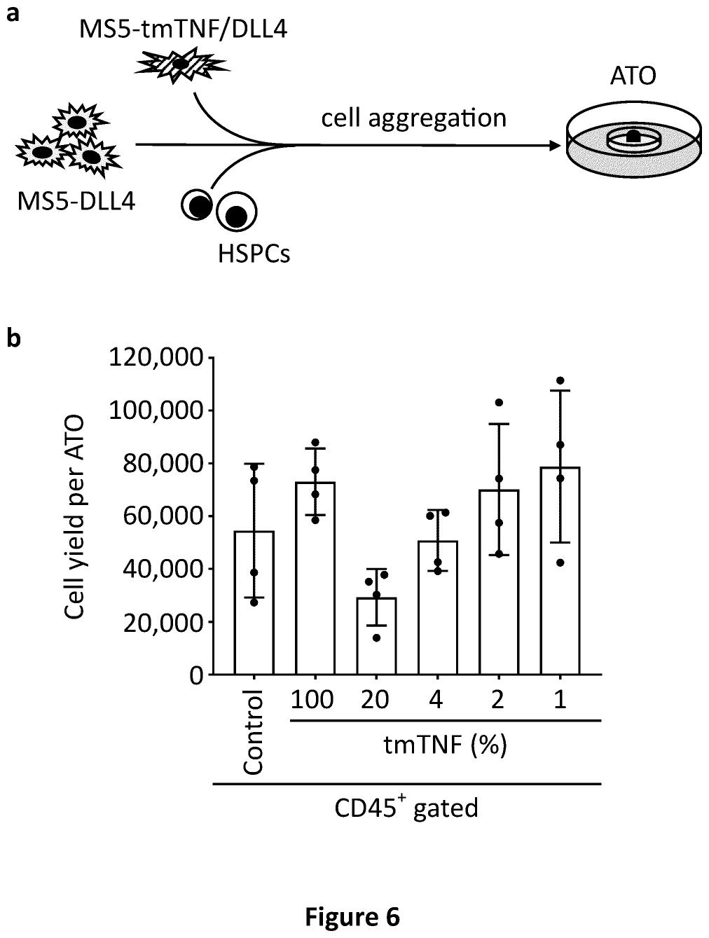

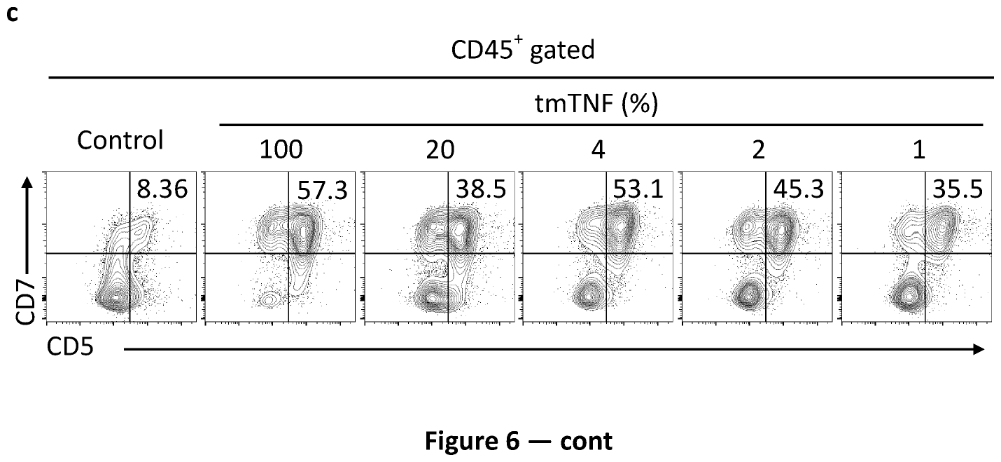

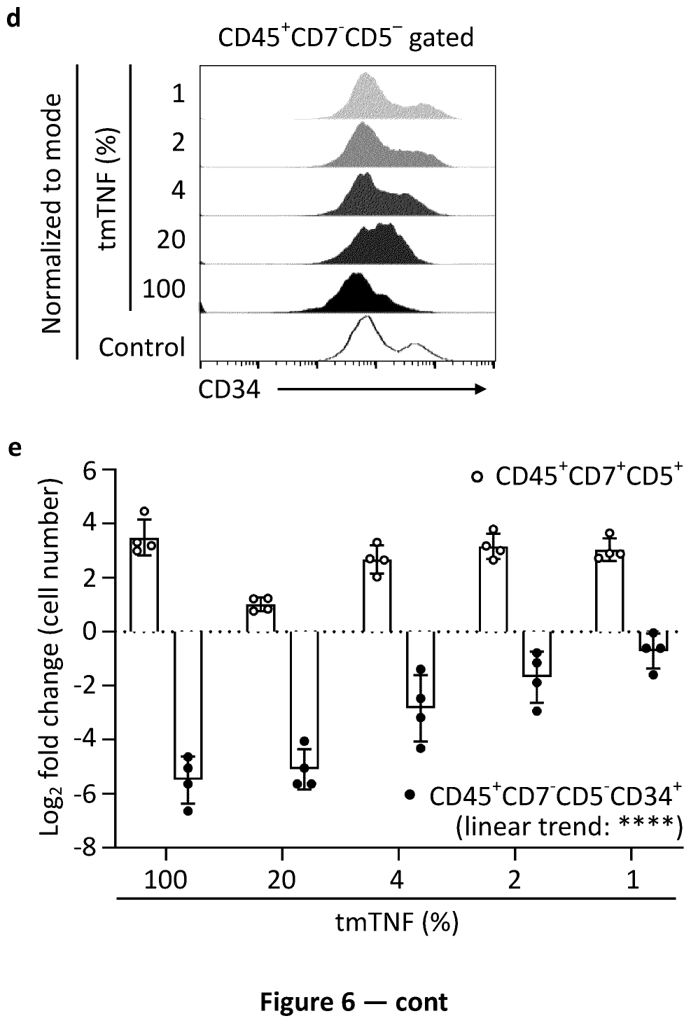

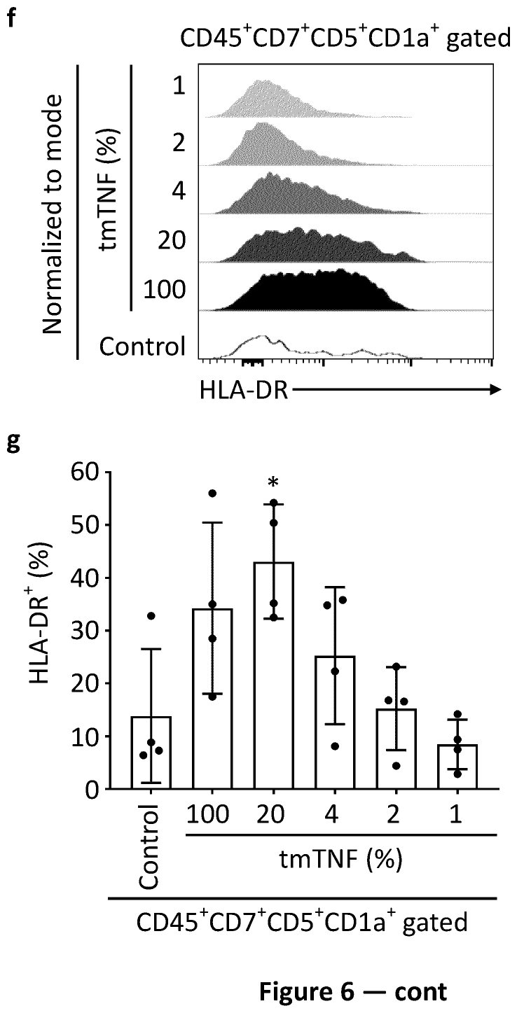

In the conventional ATO system, in vitro human T cell development is supported by the MS5 murine bone marrow stromal cell line that expresses human DLL4 (MS5-DLL4), one of the Notch ligands that induces Notch signaling in HSPCs upon cellular contact [22,33]. In order to activate TNF signaling via TNFR2, this disclosure discloses a MS5 cell line that expresses human transmembrane TNF (tmTNF) in addition to the human DLL4 (MS5-tmTNF/DLL4) (FIG. 5). It is noteworthy that tmTNF can bind to and activate both TNFR1 and TNFR2 [10]. Given that TNFR1 expression on HSPCs is downregulated during early human T cell development, this disclosure further discloses that a spatially restricted tmTNF signal minimizes the activation of TNFR1 and thereby allows preferential targeting of TNFR2. This was achieved by assembling ATOs using a combination of MS5-tmTNF/DLL4 and MS5-DLL4 at different ratios (FIG. 6A). At day 10 post culture, the presence of tmTNF signal, presented by 1 to 100% of the total MS5 stromal cells in an ATO, did not affect the cellular output compared to the control (FIG. 6B).However, unlike sTNF treatment where a higher dose limits the expansion of T cell precursors, this disclosure discloses that tmTNF signal, regardless of its intensity (1-100%), increases the generation of T cell precursors proportionally (FIG. 6C) and quantitatively (FIG. 6E). This is attributed to the activation of TNFR2. Similar to the dose-dependent activation of TNFR1 by sTNF treatment, higher intensity of tmTNF signal activates TNFR1, as indicated by the increase of aberrant HLA-DR expression on CD1a-expressing T cell precursors (FIGS. 6F and 6G), and reduces the cellular pool of undifferentiated CD34⁺ HSPCs (FIGS. 6D and 6E). The spatial restriction of tmTNF signal, at 1%, led to expansion of T cell precursors (log₂ fold change: 1% tmTNF=3.04±0.42 vs 0.25 ng/mL sTNF=2.25±0.40) without skewing the differentiation of HSPCs over proliferation (log₂ fold change: 1% tmTNF=−0.72±0.65 vs 0.25 ng/ml sTNF=−1.27±0.42). Furthermore, this disclosure shows that when tmTNF is presented by 1% of the total stromal cells in an ATO, the generated CD1a-expressing T cell precursors had the lowest expression of HLA-DR, which was similar to the control (FIGS. 6F and 6G). This further demonstrated that, by providing spatially restricted tmTNF signal at 1%, successful and selective targeting of TNFR2 occurs while the generation of T cell precursors per HSPCs has been maximized.

Using single-cell RNA sequencing technology (scRNA-seq), this disclosure reveals the global impact of TNF stimulus on HSPCs being differentiated in the ATO system. To achieve that, CD45⁺ hematopoietic cells were sorted from ATOs after 10 days of culture for scRNA-seq. Based on the transcriptomes of individual cells, 38,290 cells from the standard (control) and TNF-activated (sTNF at 0.25 ng/m or tmTNF at 1%) ATOs were grouped into 26 clusters. These clusters (0 to 25) were subsequently annotated based on their expression of cell type-specific marker genes (FIGS. 7A-7C). For example, cluster 9, 7, 5, 24, 0, 1, 2 and 14 were labelled as T cell precursors because they express T-lineage specific marker genes such as RAG2, CD1E and BCL11B (FIG. 7A) [42]. On the other hand, cluster 6, 10, 16, 11 and 20 were labelled as bi-potent natural killer (NK)/T precursors because they express both NK- (such as KLRK1 and KLRB1) and T-lineage associated genes. Consistent with the function of the ATO system [22], T cell precursors were identified as the biggest population (43.1%) among the differentiating cells (FIG. 7D). 45.6% and 41.5% of the cells labelled as T cell precursors were derived from sTNF-treated and tmTNF-spiked ATOs, respectively. This is in accordance with the above findings based on the flow cytometry analyses that both sTNF at 0.25 ng/ml and tmTNF at 1% promote the generation of T cell precursors. However, scRNA-seq analyses also identified that sTNF and tmTNF have differential impacts on the development of other minor hematopoietic populations. For example, 62.3% of the cells labelled as macrophages were derived from sTNF-treated ATOs whereas 69.5% of the cells labelled as mast cells were derived from tmTNF-spiked ATOs (FIG. 7D). This indicates that TNF signaling, mediated by sTNF and tmTNF, is not exactly identical in differentiating HSPCs. Furthermore, gene signatures for TNFR1- and TNFR2-mediated NF-κB pathway genes were evaluated and scored in all the labelled hematopoietic populations. This disclosure demonstrates that T cell precursors is the second population have the highest activation of TNFR2, which is in agreement with the induced expression of TNFR2 during their development (FIG. 7E). Importantly, cells derived from tmTNF-spiked ATOs consistently have higher activation of TNFR2 compared to that of sTNF-treated ATOs across all the labelled hematopoietic populations. Hence, sTNF (at 0.25 ng/mL) and tmTNF (at 1%) promote the generation of human T cell precursors through activation of TNFR1 and TNFR2, respectively. Finally, differential gene expression (DGE) analyses in populations of the lymphoid lineage (lymphoid precursors, T cell precursors and NK/T precursors) revealed that, compared to the control, cells derived from sTNF-treated and tmTNF-spiked ATOs have common as well as unique differentially expressed genes. This fits with the literature that activation of TNFR1 (by 0.025 ng/mL of sTNF) and TNFR2 (by 1% of tmTNF-expressing stromal cells) mediate common as well as unique downstream signaling [19]. Overall, this disclosure discloses that T cell precursors generated from sTNF-treated ATOs and tmTNF-spiked ATOS exhibit differences in their transcriptomes, although they are phenotypically similar (CD7⁺CD5⁺), consistent with their different further differentiation potential toward CD4⁺CD8b⁺ (DP) T cells.

Indeed, this disclosure further examined the developmental potential of sTNF- and tmTNF-derived T cell precursors. This is another important cell quality attribute because competent T cell precursors should be capable of differentiating further in order to generate mature T cells for therapeutic applications. To this end, at day 8 post culture, CD45⁺CD7⁺ cells were sorted from the control and TNF-activated (sTNF at 0.25 ng/m or tmTNF at 1%) ATOs for secondary culture without additional TNF stimulus (FIG. 8A). At day 13 post secondary culture, the development of DP T cells from T cell precursors was assessed (FIG. 8B). DP T cells represent an intermediate stage of T cell development where cells have passed through the β-selection checkpoint [43]. In contrast to T cell precursors derived from the control and tmTNF-spiked ATOs, sTNF-derived T cell precursors generated the least amount of DP T cells (FIG. 8). At day 25 post secondary culture, the development of CD3⁺TCRαβ⁺ and CD3⁺TCRγδ⁺ T cells was assessed (FIG. 8D). CD3⁺TCRαβ⁺ and CD3⁺TCRγδ⁺ T cells are more mature than DP T cells because they express a fully functional TCRαβ or TCRγδ complex [16]. At this stage, similar amount of T cells were generated from the control-, sTNF- and tmTNF-derived T cell precursors (FIG. 8E). Nevertheless, the TCR diversity of CD3⁺TCRαβ⁺ T cells that are developed from the sTNF-derived T cell precursors might be restricted due to the impaired development at the DP stage. Hence, this disclosure reveals that the quality of T cell precursors derived from sTNF-treated and tmTNF-spiked ATOs are different in terms of their developmental potential and gene expression.

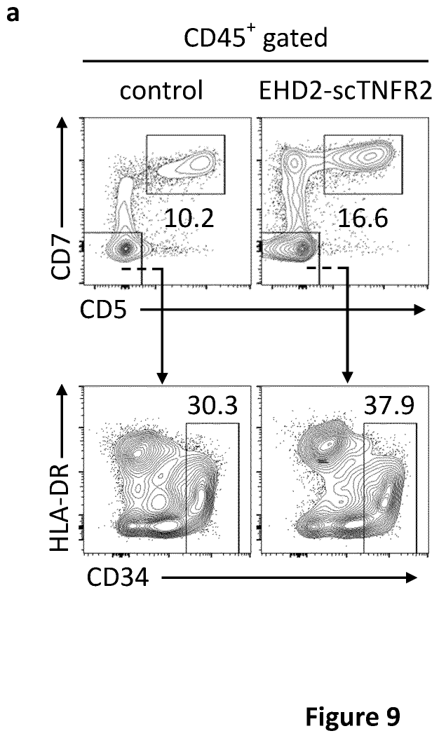

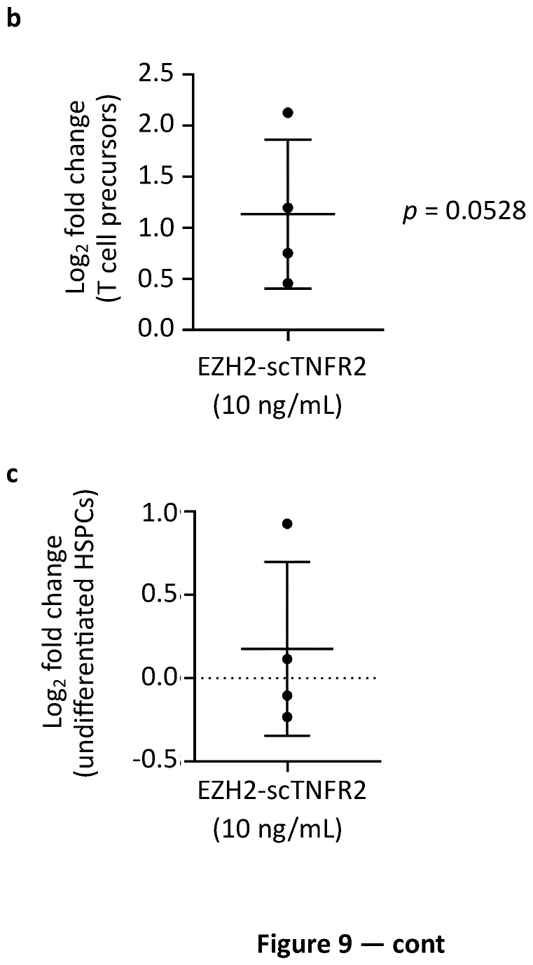

To further illustrate the importance of targeting TNFR2 in enhancing the generation of human T cell precursors, a TNFR2-selective agonist (EHD2-scTNFR2) was tested [19,20]. At day 10 post ATO culture, EHD2-scTNFR2 increased the frequency (FIG. 9A) and cell number (FIG. 9B) of T cell precursors compared to the control. Importantly, in consistent with tmTNF-expressing stromal cells (FIG. 6E), EHD2-scTNFR2 did not deplete the pool of undifferentiated HSPCs (FIG. 9C). Hence, this disclosure discloses that, besides tmTNF-expressing stromal cells, pharmacological activation of TNFR2 can be achieved by using a TNFR2-selective agonist in order to maximize the generation of human T cell precursors in vitro.

### REFERENCES

1. Awong, G. et al. Characterization in vitro and engraftment potential in vivo of human progenitor T cells generated from hematopoietic stem cells. Blood 114, 972-982, doi:10.1182/blood-2008-10-187013 (2009).
2. Reimann, C. et al. Human T-lymphoid progenitors generated in a feeder-cell-free Delta-like-4 culture system promote T-cell reconstitution in NOD/SCID/gammac(−/−) mice. Stem Cells 30, 1771-1780, doi: 10.1002/stem. 1145 (2012).
3. Shukla, S. et al. Progenitor T-cell differentiation from hematopoietic stem cells using Delta-like-4 and VCAM-1. Nat Methods 14, 531-538, doi: 10.1038/nmeth.4258 (2017).
4. Weekx, S. F. et al. Generation of T cells from adult human hematopoietic stem cells and progenitors in a fetal thymic organ culture system: stimulation by tumor necrosis factor-alpha. Blood 95, 2806-2812 (2000).
5. Dos Santos Schiavinato, J. L. et al. TNF-alpha and Notch signaling regulates the expression of HOXB4 and GATA3 during early T lymphopoiesis. In Vitro Cell Dev Biol Anim 52, 920-934, doi:10.1007/s11626-016-0055-8 (2016).
6. Smits, K. et al. Tumor necrosis factor promotes T-cell at the expense of B-cell lymphoid development from cultured human CD34+ cord blood cells. Exp Hematol 35, 1272-1278, doi:10.1016/j.exphem.2007.04.009 (2007).
7. Edgar, J. M. & Zandstra, P. W. Inflammatory Cytokines Regulate T-Cell Development from Blood Progenitor Cells in a Stage and Dose-Specific Manner. bioRxiv, 2021.01.18.427186, doi: 10.1101/2021.01.18.427186 (2021).
8. Kriegler, M., Perez, C., DeFay, K., Albert, I. & Lu, S. D. A novel form of TNF/cachectin is a cell surface cytotoxic transmembrane protein: ramifications for the complex physiology of TNF. Cell 53, 45-53, doi: 10.1016/0092-8674(88)90486-2 (1988).
9. Black, R. A. et al. A metalloproteinase disintegrin that releases tumor-necrosis factor-alpha from cells. Nature 385, 729-733, doi: 10.1038/385729a0 (1997).
10. Grell, M. et al. The transmembrane form of tumor necrosis factor is the prime activating ligand of the 80 kDa tumor necrosis factor receptor. Cell 83, 793-802, doi: 10.1016/0092-8674(95)90192-2 (1995).
11. Grell, M., Wajant, H., Zimmermann, G. & Scheurich, P. The type 1 receptor (CD120a) is the high-affinity receptor for soluble tumor necrosis factor. Proc Natl Acad Sci USA 95, 570-575, doi:10.1073/pnas.95.2.570 (1998).
12. Dybedal, I., Bryder, D., Fossum, A., Rusten, L. S. & Jacobsen, S. E. Tumor necrosis factor (TNF)-mediated activation of the p55 TNF receptor negatively regulates maintenance of cycling reconstituting human hematopoietic stem cells. Blood 98, 1782-1791, doi:10.1182/blood.v98.6.1782 (2001).
13. Senyuk, V., Patel, P., Mahmud, N. & Rondelli, D. Blockade of TNFalpha to Improve Human CD34+ Cell Repopulating Activity in Allogeneic Stem Cell Transplantation. Front Immunol 9, 3186, doi: 10.3389/fimmu.2018.03186 (2018).
14. Civin, C. I. et al. Antigenic analysis of hematopoiesis. III. A hematopoietic progenitor cell surface antigen defined by a monoclonal antibody raised against KG-1a cells. Journal of Immunology 133, 157-165 (1984).
15. Ivanovs, A. et al. Human haematopoietic stem cell development: from the embryo to the dish. Development 144, 2323-2337, doi: 10.1242/dev.134866 (2017).
16. Taghon, T., Waegemans, E. & Van de Walle, I. Notch signaling during human T cell development. Curr Top Microbiol Immunol 360, 75-97, doi: 10.1007/82_2012_230 (2012).
17. De Smedt, M. et al. Active form of Notch imposes T cell fate in human progenitor cells. J Immunol 169, 3021-3029, doi: 10.4049/jimmunol. 169.6.3021 (2002).
18. Martin-Gayo, E. et al. Spatially restricted JAG1-Notch signaling in human thymus provides suitable DC developmental niches. J Exp Med 214, 3361-3379, doi:10.1084/jem.20161564 (2017).
19. Dong, Y. et al. Essential protective role of tumor necrosis factor receptor 2 in neurodegeneration. Proc Natl Acad Sci USA 113, 12304-12309 (2016).
20. Fischer, R., Kontermann, R. E. & Pfizenmaier, K. Selective Targeting of TNF Receptors as a Novel Therapeutic Approach. Front Cell Dev Biol 8, 401, doi:10.3389/fcell.2020.00401 (2020).
21. Barrett, D. M., Grupp, S. A. & June, C. H. Chimeric Antigen Receptor- and TCR-Modified T Cells Enter Main Street and Wall Street. J Immunol 195, 755-761, doi: 10.4049/jimmunol.1500751 (2015).
22. Seet, C. S. et al. Generation of mature T cells from human hematopoietic stem and progenitor cells in artificial thymic organoids. Nat Methods 14, 521-530, doi: 10.1038/nmeth.4237 (2017).
23. Montel-Hagen, A. et al. Organoid-Induced Differentiation of Conventional T Cells from Human Pluripotent Stem Cells. Cell Stem Cell 24, 376-389 e378, doi:10.1016/j.stem.2018.12.011 (2019).
24. De Smedt, M., Hoebeke, I. & Plum, J. Human bone marrow CD34+ progenitor cells mature to T cells on OP9-DL 1 stromal cell line without thymus microenvironment. Blood Cells Mol Dis 33, 227-232, doi: 10.1016/j.bcmd.2004.08.007 (2004).
25. Schmitt, T. M. & Zuniga-Pflucker, J. C. Induction of T cell development from hematopoietic progenitor cells by delta-like-1 in vitro. Immunity 17, 749-756, doi: 10.1016/s1074-7613(02)00474-0 (2002).
26. Maney, N. J., Reynolds, G., Krippner-Heidenreich, A. & Hilkens, C. M. U. Dendritic cell maturation and survival are differentially regulated by TNFR1 and TNER2. J Immunol 193, 4914-4923, doi: 10.4049/jimmunol. 1302929 (2014).
27. Itoh, K. et al. Reproducible establishment of hemopoietic supportive stromal cell lines from murine bone marrow. Exp Hematol 17, 145-153 (1989).
28. Van de Walle, I. et al. Jagged2 acts as a Delta-like Notch ligand during early hematopoietic cell fate decisions. Blood 117, 4449-4459, doi: 10.1182/blood-2010-06-290049 (2011).
29. Van de Walle, I. et al. An early decrease in Notch activation is required for human TCR-alphabeta lineage differentiation at the expense of TCR-gammadelta T cells. Blood 113, 2988-2998, doi: 10.1182/blood-2008-06-164871 (2009).
30. Williams, C. K., Li, J. L., Murga, M., Harris, A. L. & Tosato, G. Upregulation of the Notch ligand Delta-like 4 inhibits VEGF-induced endothelial cell function. Blood 107, 931-939, doi: 10.1182/blood-2005-03-1000 (2006).
31. Taghon, T. et al. Enforced expression of GATA-3 severely reduces human thymic cellularity. J Immunol 167, 4468-4475, doi: 10.4049/jimmunol. 167.8.4468 (2001).
32. Taghon, T. et al. HOX-A10 regulates hematopoietic lineage commitment: evidence for a monocyte-specific transcription factor. Blood 99, 1197-1204, doi: 10.1182/blood.v99.4.1197 (2002).
33. Dolens, A. C. et al. Distinct Notch1 and BCL11B requirements mediate human gammadelta/alphabeta T cell development. EMBO Rep 21, e49006, doi: 10.15252/embr.201949006 (2020).
34. Plum, J., De Smedt, M., Defresne, M. P., Leclercq, G. & Vandekerckhove, B. Human CD34+ fetal liver stem cells differentiate to T cells in a mouse thymic microenvironment. Blood 84, 1587-1593 (1994).
35. Butler, A., Hoffman, P., Smibert, P., Papalexi, E. & Satija, R. Integrating single-cell transcriptomic data across different conditions, technologies, and species. Nat Biotechnol 36, 411-420 (2018).
36. Mclnnes, L., Healy, J. & Melville, J. UMAP: Uniform Manifold Approximation and Projection for Dimension Reduction. arXiv preprint arXiv: 1802.03426 (2018).
37. Andreatta, M. & Carmona, S. J. UCell: Robust and scalable single-cell gene signature scoring. Comput Struct Biotechnol J 19, 3796-3798 (2021).
38. Liberzon, A., Subramanian, A., Pinchback, R., Thorvaldsdóttir, H., Tamayo, P. & Mesirov, J. P. Bioinformatics 27, 1739-1740 (2011).
39. Zielske, S. P. & Braun, S. E. Cytokines: value-added products in hematopoietic stem cell gene therapy. Mol Ther 10, 211-219, doi:10.1016/j.ymthe.2004.05.025 (2004).
40. Van de Walle, I. et al. GATA3 induces human T-cell commitment by restraining Notch activity and repressing NK-cell fate. Nat Commun 7, 11171, doi:10.1038/ncomms11171 (2016).
41. Aggarwal, S., Gollapudi, S. & Gupta, S. Increased TNF-alpha-induced apoptosis in lymphocytes from aged humans: changes in TNF-alpha receptor expression and activation of caspases. J Immunol 162, 2154-2161 (1999).
42. Cytlak, U. et al. Differential IRF8 transcription factor requirement defines two pathways of dendritic cell development in humans. Immunity 53, 353-370.e8 (2020).
43. Lavaert, M. et al. Integrated scRNA-seq identifies human postnatal thymus seeding progenitors and regulatory dynamics of differentiating immature thymocytes. Immunity 52, 1088-1104.e6 (2020).
44. Taghon, T. et al. Notch signaling is required for proliferation but not for differentiation at a well-defined beta-selection checkpoint during human T-cell development. Blood 113, 3254-3263 (2009).

1.-13. (canceled)

14. An in vitro method for generating T-cell precursors from hematopoietic stem and progenitor cells (HSPCs), the method comprising:
contacting HSPCs with an agonist for tumor necrosis factor receptor 2 (TNFR2) so as to generate T-cell precursors from the HSPCs.

15. The method according to claim 14, wherein the T-cell precursors are CD5+ and CD7+ T-cell precursors.

16. The method according to claim 14, wherein the HSPCs are derived from embryonic or induced pluripotent stem cells, cord blood, bone marrow, or mobilized peripheral blood.

17. The method according to claim 14, wherein the T-cell precursors are chimeric antigen receptor or T-cell receptor engineered T cells from primary or pluripotent stem cell-derived HSPCs.

18. The method according to claim 14, wherein the agonist for TNFR2 is transmembrane tumor necrosis factor-alpha (TNF-α).

19. The method according to claim 14, wherein a pool of undifferentiated CD34+ HSPCs is maintained.

20. The method according to claim 14, wherein the agonist improves development potential of the T-cell precursors.

21. An in vitro system for generating T-cell precursors comprising:
hematopoietic stem and progenitor cells, and
an agonist for tumor necrosis factor receptor 2 (TNFR2).

22. The system of claim 21, wherein the system is an artificial thymic organoid system comprising stromal cells that express agonist for TNFR2.

23. The system of claim 22, wherein stromal cells that express an agonist for TNFR2 constitute about 1% of the total amount of stromal cells.

24. The system of claim 22, wherein the stromal cell that expresses an agonist for TNFR2 is a MS5 murine bone marrow stromal cell line that expresses human transmembrane tumor necrosis factor-alpha (TNF-α).

25. The system of claim 24, wherein the MS5 murine bone marrow stromal cell line that expresses human transmembrane TNF-α also expresses Notch ligand human DLL4.

26. The system of claim 25, comprising both:
MS5 murine bone marrow stromal cell line that expresses both human transmembrane (TNF-α), and
Notch ligand human DLL4 and a MS5 murine bone marrow stromal cell line that expresses human DLL4,
wherein the amounts of both cell lines are present in the system at a ratio of 1:99, respectively.

27. An in vitro method for generating T-cell precursors from hematopoietic stem and progenitor cells (HSPCs), the method comprising:
contacting HSPCs derived from embryonic or induced pluripotent stem cells, cord blood, bone marrow, or mobilized peripheral blood with an agonist for tumor necrosis factor receptor 2 (TNFR2), wherein the agonist for TNFR2 is transmembrane tumor necrosis factor-alpha (TNF-α),
so as to generate T-cell precursors from the HSPCs,
wherein the T-cell precursors are CD5+ and CD7+ T-cell precursors or chimeric antigen receptor or T-cell receptor engineered T cells from primary or pluripotent stem cell-derived HSPCs.

28. The method according to claim 27, wherein a pool of undifferentiated CD34+ HSPCs is maintained.
